(19) United States
(12) Patent Application Publication
(10) Pub. No.: US 2026/0183250 A1
(43) Pub. Date: Jul. 2, 2026

(54) SALBUTAMOL DELIVERY COMPOSITIONS, DEVICES AND METHODS

(71) Applicant: Solstice Advanced Materials US, Inc., Morris Plains, NJ (US)

(72) Inventors: Erik Boldt, Lancaster, NY (US); Gregory Laurence Smith, Buffalo, NY (US)

(21) Appl. No.: 19/434,305

(22) Filed: Dec. 29, 2025

Related U.S. Application Data
(60) Provisional application No. 63/740,832, filed on Dec. 31, 2024.

(51) Int. Cl. A61K 31/137; A61K 9/00; A61K 47/06; A61K 47/10

(52) U.S. Cl. CPC A61K 31/137; A61K 9/008; A61K 47/06; A61K 47/10

(57) ABSTRACT

Disclosed are medicinal compositions, and devices, methods and systems which use same, comprising a carrier and at least one medicinally active compound, said carrier comprising at least 1234ze(E), ethanol and polyethylene glycol.

### CROSS REFERENCE TO RELATED APPLICATIONS

This application claims priority benefit from U.S. Provisional Application No. 63/740,832 filed Dec. 31, 2024, the entire contents of which are hereby incorporated by reference.

### FIELD OF THE INVENTION

This invention relates to medicament delivery compositions, systems, devices and methods. In particular aspects, this invention relates to medicinal aerosol compositions, methods and devices used for metered dose delivery of salbutamol.

### BACKGROUND OF THE INVENTION

Metered dose inhalers (MDIs) have long been used to deliver medicaments, such as bronchodilator drugs and steroids, to the areas of patients needing treatment. Compared with oral administration of bronchodilators, inhalation therapy using MDIs frequently has the advantage of relatively rapid onset of action and relatively low instance of systemic side effects.

MDIs may be used to deliver medicaments in a solubilized form or as a suspension. When MDIs use a relatively high vapor pressure propellant to carry and expel aerosolized droplets containing an API into the respiratory tract, they are known as pressurized MDIs (sometimes referred to herein as pMDIs). The propellant/carrier used in pMDIs for the active pharmaceutical ingredient (sometimes referred to herein as “API”) must be safe for patients' use and be pharmaceutically acceptable. The API to be delivered by a pMDI in many applications is provided as a suspension of particulates dispersed within a carrier which helps to form a suspension and/or otherwise carry the active ingredient.

Asthma is one condition that has frequently been treated using of MDIs. Asthma has been described as a chronic disease that involves inflammation of the pulmonary airways and bronchial hyperresponsiveness that results in the clinical expression of a lower airway obstruction that usually is reversible. The pathophysiology of asthma or related disorders involves bronchoconstriction resulting from bronchial smooth muscle spasm and airway inflammation with mucosal edema. Treatment of asthma and other related disorders (including Chronic Obstructive Pulmonary Disease (COPD)) has included the administration of β-2 agonists, also known as, β-2 adrenoreceptor agonists. Such β-2 adrenoreceptor agonists are known to provide a bronchodilator effect to patients, resulting in relief from the symptoms of breathlessness. More particularly, β-2 adrenoreceptor agonists have been shown to increase the conductance of potassium channels in airway muscle cells, leading to membrane hyperpolarization and relaxation. Salbutamol is a short-acting β-2 adrenoreceptor and has been recommended and used for the relief of acute asthma symptoms.

Several challenges exist in connection with efforts to provide new MDIs in general, and suspension-based pMDI propellants for the delivery of salbutamol in particular. For example, while there are advantages to delivery of the API as fine particles suspended in a propellant, there are also difficult technical challenges associated with such delivery methods and systems. For example, when a formulation is designed for delivering an API in suspension, the use of a carrier which increases the solubility of the API in the formulation can detrimentally facilitate particle growth. Such a problem is disclosed in connection with the use of ethanol in combination with HFO-1234ze(E) for the delivery of salbutamol sulfate in suspension formulations. See WO 2023/039103, which discloses the use of HFO-1234ze(E) and ethanol, where ethanol is present in amounts of between 3% to 8%, or 2% to 5% in a suspension formulation which includes an API comprising fluticasone, salbutamol or mometasone.

Applicants have come to appreciate that while the use of HFO-1234ze(E) together with ethanol as the carrier for salbutamol in suspension has been disclosed, at least two significant disadvantages can be associated with such formulations. In particular, applicants have found that such formulations can exhibit an undesirably high level of dose variation and/or that such formulation can produce a mean measured dosage that is undesirably above or below the target dosage.

Thus, notwithstanding the disclosures as mentioned above, applicants have come to recognize the need for delivery compositions, systems, devices and methods for salbutamol that at once provide a delivered dose that has a relatively low dosage variability.

### SUMMARY

Applicants have found that many of the shortcomings of the prior compositions can be overcome and/or that many of the above-noted needs or other needs can be satisfied by pharmaceutical compositions of the present invention and the use thereof in pMDIs and by the present inhalation delivery methods.

The present invention includes pharmaceutical compositions comprising:
a. fine particles of salbutamol; and
b. a carrier in which said fine particles of salbutamol are suspended, said carrier comprising: (i) from about 90% by weight to less than 98% by weight of HFO-1234ze(E); (ii) from greater than 2% by weight to about 10% by weight of ethanol; and (iii) from about 0.005% by weight to about 0.01% by weight of polyethylene glycol.

For the purposes of convenience, pharmaceutical compositions according to this paragraph are referred to as Pharmaceutical Composition 1A1.

The present invention includes pharmaceutical compositions comprising:
a. fine particles of salbutamol sulfate; and
b. a carrier in which said fine particles of salbutamol sulfate are suspended, said carrier comprising: (i) from about 90% by weight to less than 98% by weight of HFO-1234ze(E); (ii) from greater than 2% by weight to about 10% by weight of ethanol; and (iii) from about 0.005% by weight to about 0.01% by weight of polyethylene glycol.

For the purposes of convenience, pharmaceutical compositions according to this paragraph are referred to as Pharmaceutical Composition 1A2.

The present invention includes pharmaceutical compositions comprising:
a. fine particles of salbutamol; and
b. a carrier in which said fine particles of salbutamol are suspended, said carrier comprising: (i) from about 90% by weight to less than 98% by weight of HFO-1234ze(E); (ii) from greater than 2% by weight to about 10% by weight of ethanol; and (iii) from about 0.005% by weight to about 0.01% by weight of polyethylene glycol having a molecular weight of from about 300 to about 1500.

For the purposes of convenience, pharmaceutical compositions according to this paragraph are referred to as Pharmaceutical Composition 1A3.

The present invention includes pharmaceutical compositions comprising:
a. fine particles of salbutamol sulfate; and
b. a carrier in which said fine particles of salbutamol sulfate are suspended, said carrier comprising: (i) from about 90% by weight to less than 98% by weight of HFO-1234ze(E); (ii) from greater than 2% by weight to about 10% by weight of ethanol; and (iii) from about 0.005% by weight to about 0.01% by weight of polyethylene glycol having a molecular weight of from about 300 to about 1200.

For the purposes of convenience, pharmaceutical compositions according to this paragraph are referred to as Pharmaceutical Composition 1A4.

The present invention includes pharmaceutical compositions comprising:
a. fine particles of salbutamol;
b. a carrier in which said fine particles of salbutamol are suspended, said carrier comprising: (i) from about 90% by weight to less than 98% by weight of HFO-1234ze(E); (ii) from greater than 2% by weight to about 10% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 1A5.

The present invention includes pharmaceutical compositions comprising:
a. fine particles of salbutamol sulfate; and
b. a carrier in which said fine particles of salbutamol sulfate are suspended, said carrier comprising: (i) from about 90% by weight to less than 98% by weight of HFO-1234ze(E); (ii) from greater than 2% by weight to about 10% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 1A6.

The present invention includes pharmaceutical compositions consisting essentially of:
a. fine particles of salbutamol sulfate; and
b. a carrier in which said fine particles of salbutamol are suspended, said carrier comprising: (i) from about 90% by weight to less than 98% by weight of HFO-1234ze(E); (ii) from greater than 2% by weight to about 10% by weight of ethanol; and from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 1A7.

The present invention includes pharmaceutical compositions consisting essentially of:
a. fine particles of salbutamol sulfate; and
b. a carrier in which said fine particles of salbutamol sulfate are suspended, said carrier comprising: (i) from about 90% by weight to less than 98% by weight of HFO-1234ze(E); (ii) from greater than 2% by weight to about 10% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 1A8.

The present invention includes pharmaceutical compositions consisting of:
a. fine particles of salbutamol sulfate; and
b. a carrier in which said fine particles of salbutamol are suspended, said carrier comprising: (i) from about 90% by weight to less than 98% by weight of HFO-1234ze(E); (ii) from greater than 2% by weight to about 10% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 1A9.

The present invention includes pharmaceutical compositions consisting of:
a. fine particles of salbutamol sulfate; and
b. a carrier in which said fine particles of salbutamol sulfate are suspended, said carrier comprising: (i) from about 90% by weight to less than 98% by weight of HFO-1234ze(E); (ii) from greater than 2% by weight to about 10% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 1A10.

The present invention includes pharmaceutical compositions comprising:
a. fine particles of salbutamol sulfate; and
b. a carrier in which said fine particles of salbutamol sulfate are suspended, said carrier comprising: (i) from about 91% by weight to about 97% by weight of HFO-1234ze(E); (ii) from about 3% by weight to about 9% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 2A.

The present invention includes pharmaceutical compositions consisting essentially of:
a. fine particles of salbutamol sulfate; and
b. a carrier in which said fine particles of salbutamol sulfate are suspended, said carrier comprising: (i) from about 91% by weight to about 97% by weight of HFO-1234ze(E); (ii) from about 3% by weight to about 9% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 2B.

The present invention includes pharmaceutical compositions consisting of:
a. fine particles of salbutamol sulfate; and
b. a carrier in which said fine particles of salbutamol sulfate are suspended, said carrier comprising: (i) from about 91% by weight to about 97% by weight of HFO-1234ze(E); (ii) from about 3% by weight to about 9% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 2C.

The present invention includes pharmaceutical compositions comprising:
a. fine particles of salbutamol sulfate; and
b. a carrier in which said fine particles of salbutamol sulfate are suspended, said carrier comprising: (i) from about 91.5% by weight to about 96.5% by weight of HFO-1234ze(E); (ii) from about 3.5% by weight to about 8.5% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 3A.

The present invention includes pharmaceutical compositions consisting essentially of:
a. fine particles of salbutamol sulfate; and
b. a carrier in which said fine particles of salbutamol sulfate are suspended, said carrier comprising: (i) from about 91.5% by weight to about 96.5% by weight of HFO-1234ze(E); (ii) from about 3.5% by weight to about 8.5% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 3B.

The present invention includes pharmaceutical compositions consisting of:
a. fine particles of salbutamol sulfate; and
b. a carrier in which said fine particles of salbutamol sulfate are suspended, said carrier comprising: (i) from about 91.5% by weight to about 96.5% by weight of HFO-1234ze(E); (ii) from about 3.5% by weight to about 8.5% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 3C.

The present invention includes pharmaceutical compositions comprising:
a. fine particles of salbutamol sulfate; and
b. a carrier in which said fine particles of salbutamol sulfate are suspended, said carrier comprising: (i) from about 92% by weight to about 96% by weight of HFO-1234ze(E); (ii) from about 4% by weight to about 8% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 4A.

The present invention includes pharmaceutical compositions consisting essentially of:
a. fine particles of salbutamol sulfate; and
b. a carrier in which said fine particles of salbutamol sulfate are suspended, said carrier comprising: (i) from about 92% by weight to about 96% by weight of HFO-1234ze(E); (ii) from about 4% by weight to about 8% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 4B.

The present invention includes pharmaceutical compositions consisting of:
a. fine particles of salbutamol sulfate; and
b. a carrier in which said fine particles of salbutamol sulfate are suspended, said carrier comprising: (i) from about 92% by weight to about 96% by weight of HFO-1234ze(E); (ii) from about 4% by weight to about 8% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 4C.

The present invention includes pharmaceutical compositions comprising:
a. fine particles of salbutamol sulfate; and
b. a carrier in which said fine particles of salbutamol sulfate are suspended, said carrier comprising: (i) from about 93% by weight to about 96% by weight of HFO-1234ze(E); (ii) from about 4% by weight to about 7% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 5A.

The present invention includes pharmaceutical compositions consisting essentially of:
a. fine particles of salbutamol sulfate; and
b. a carrier in which said fine particles of salbutamol sulfate are suspended, said carrier comprising: (i) from about 93% by weight to about 96% by weight of HFO-1234ze(E); (ii) from about 4% by weight to about 7% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 5B.

The present invention includes pharmaceutical compositions consisting of:
a. fine particles of salbutamol sulfate; and
a. a carrier in which said fine particles of salbutamol sulfate are suspended, said carrier comprising: (i) from about 93% by weight to about 96% by weight of HFO-1234ze(E); (ii) from about 4% by weight to about 7% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 5C.

The present invention includes pharmaceutical compositions comprising:
a. fine particles of salbutamol sulfate; and
b. a carrier in which said fine particles of salbutamol sulfate are suspended, said carrier comprising: (i) from about 94% by weight to about 96% by weight of HFO-1234ze(E); (ii) from about 4% by weight to about 6% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 6A.

The present invention includes pharmaceutical compositions consisting essentially of:
a. fine particles of salbutamol sulfate; and
b. a carrier in which said fine particles of salbutamol sulfate are suspended, said carrier comprising: (i) from about 94% by weight to about 96% by weight of HFO-1234ze(E); (ii) from about 4% by weight to about 6% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 6B.

The present invention includes pharmaceutical compositions consisting of:
a. particles of salbutamol sulfate; and
b. a carrier in which said fine particles of salbutamol sulfate are suspended, said carrier comprising: (i) from about 94% by weight to about 96% by weight of HFO-1234ze(E); (ii) from about 4% by weight to about 6% by weight of ethanol; and from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 6C.

The present invention includes pharmaceutical compositions comprising:
a. fine particles of salbutamol sulfate; and
b. a carrier in which said fine particles of salbutamol sulfate are suspended, said carrier comprising: (i) about 95% by weight of HFO-1234ze(E); (ii) about 5% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 7A.

The present invention includes pharmaceutical compositions consisting essentially of:
a. fine particles of salbutamol sulfate; and
b. a carrier in which said fine particles of salbutamol sulfate are suspended, said carrier comprising: (i) about 95% by weight of HFO-1234ze(E); (ii) about 5% by weight of ethanol; and (iii) about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 7B.

The present invention includes pharmaceutical compositions consisting of:
a. fine particles of salbutamol sulfate; and
b. a carrier in which said fine particles of salbutamol sulfate are suspended, said carrier comprising: (i) about 95% by weight of HFO-1234ze(E); (ii) about 5% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 7C.

The present invention includes pharmaceutical compositions comprising:
a. from about 2.5 mg/mL to less than about 4.5 mg/mL of fine particles of salbutamol sulfate; and
b. a carrier in which said fine particles of salbutamol are suspended, said carrier comprising: (i) from greater than about 90% by weight to less than 98% by weight of HFO-1234ze(E); (ii) from greater than 2% by weight to about 10% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 8A.

The present invention includes pharmaceutical compositions comprising:
a. from about 2.5 mg/mL to less than about 4.5 mg/mL of fine particles of salbutamol sulfate; and
b. a carrier in which said fine particles of salbutamol sulfate are suspended, said carrier comprising: (i) from greater than about 90% by weight to less than 98% by weight of HFO-1234ze(E); (ii) from greater than 2% by weight to about 10% by weight of ethanol; and (iii) from about 0.005% by weight to about 0.01% by weight of polyethylene glycol.

For the purposes of convenience, pharmaceutical compositions according to this paragraph are referred to as Pharmaceutical Composition 8B.

The present invention includes pharmaceutical compositions comprising:
a. from about 2.5 mg/mL to less than about 4.5 mg/mL of fine particles of salbutamol sulfate; and
b. a carrier in which said fine particles of salbutamol are suspended, said carrier comprising: (i) from greater than about 90% by weight to less than 98% by weight of HFO-1234ze(E); (ii) from greater than 2% by weight to about 10% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 9A.

The present invention includes pharmaceutical compositions comprising:
a. from about 2.5 mg/mL to less than about 4.5 mg/mL of fine particles of salbutamol sulfate; and
b. a carrier in which said fine particles of salbutamol sulfate are suspended, said carrier comprising: (i) from greater than about 90% by weight to less than 98% by weight of HFO-1234ze(E); (ii) from greater than 2% by weight to about 10% by weight of ethanol; and (iii) from about 0.005% by weight to about 0.01% by weight of polyethylene glycol having a molecular weight of from about 400 to about 1000.

For the purposes of convenience, pharmaceutical compositions according to this paragraph are referred to as Pharmaceutical Composition 9B.

The present invention also includes methods for delivering a dose of salbutamol comprising:
a. providing a pMDI comprising a container containing: (1) a carrier comprising: (i) from greater than about 90% by weight to less than 98% by weight of HFO-1234ze(E); (ii) from greater than 2% by weight to less than about 10% by weight of ethanol; and (iii) from about 0.005% by weight to about 0.01% by weight of polyethylene glycol; and (2) fine particles of salbutamol suspended in one or more of said carrier components; and
b. actuating said pMDI to produce an aerosol spray of said salbutamol.

For the purposes of convenience, methods according to this paragraph are referred to as Pharmaceutical Delivery Method 1A.

The present invention also includes methods for delivering a dose of salbutamol sulfate comprising:
a. providing a pMDI comprising a container containing: (1) a carrier comprising: (i) from greater than about 90% by weight to less than 98% by weight of HFO-1234ze(E); and (ii) from greater than 2% by weight to less than about 10% by weight of ethanol; and (iii) from about 0.005% by weight to about 0.01% by weight of polyethylene glycol; and (2) fine particles of salbutamol sulfate suspended in one or more of said carrier components; and
b. actuating said pMDI to produce an aerosol spray of said salbutamol sulfate.

For the purposes of convenience, methods according to this paragraph are referred to as Pharmaceutical Delivery Method 1B.

The present invention also includes methods for delivering a target dose of salbutamol comprising:
a. providing a pMDI comprising a container containing: (1) a carrier comprising: (i) from greater than about 90% by weight to less than 98% by weight of HFO-1234ze(E); (ii) from greater than 2% by weight to less than about 10% by weight of ethanol and (iii) from about 0.005% by weight to about 0.01% by weight of polyethylene glycol; and (2) fine particles of salbutamol suspended in one or more of said carrier components; and
b. actuating said MDI to produce an aerosol spray of said salbutamol, wherein said spray has an RSD % of not greater than 30%.

For the purposes of convenience, methods according to this paragraph are referred to as Pharmaceutical Delivery Method 2A.

The present invention also includes methods for delivering a target dose of salbutamol sulfate comprising:
a. providing a pMDI comprising a container containing: (1) a carrier comprising: (i) from greater than about 90% by weight to less than 98% by weight of HFO-1234ze(E); (ii) from greater than 2% by weight to less than about 10% by weight of ethanol and (iii) from about 0.005% by weight to about 0.01% by weight of polyethylene glycol; and (2) fine particles of salbutamol sulfate suspended in one or more of said carrier components; and
b. actuating said MDI to produce an aerosol spray of said salbutamol sulfate, wherein said spray has an RSD % of not greater than 30% and a delivered dose of at least about 150 μg/actuation.

For the purposes of convenience, methods according to this paragraph are referred to as Pharmaceutical Delivery Method 2B.

The present invention also includes methods for delivering a target dose of salbutamol comprising:
a. providing a pMDI comprising a container containing: (1) a carrier comprising: (i) from greater than about 90% by weight to less than 98% by weight of HFO-1234ze(E); (ii) from greater than 2% by weight to less than about 10% by weight of ethanol and (iii) from about 0.005% by weight to about 0.01% by weight of polyethylene glycol; and (2) fine particles of salbutamol suspended in one or more of said carrier components; and
b. actuating said MDI to produce an aerosol spray of said salbutamol, wherein said spray has an RSD % of not greater than 25% and a delivered dose of at least about 150 μg/actuation.

For the purposes of convenience, methods according to this paragraph are referred to as Pharmaceutical Delivery Method 3A.

The present invention also includes methods for delivering a target dose of salbutamol sulfate comprising:
c. providing a pMDI comprising a container containing: (1) a carrier comprising: (i) from greater than about 90% by weight to less than 98% by weight of HFO-1234ze(E); (ii) from greater than 2% by weight to less than about 10% by weight of ethanol and (iii) from about 0.005% by weight to about 0.01% by weight of polyethylene glycol; and (2) fine particles of salbutamol sulfate suspended in one or more of said carrier components; and
d. actuating said MDI to produce an aerosol spray of said salbutamol sulfate, wherein said spray has an RSD % of not greater than 25% and a delivered dose of at least about 150 μg/actuation.

For the purposes of convenience, methods according to this paragraph are referred to as Pharmaceutical Delivery Method 3B.

The present invention also includes methods for delivering a target dose of salbutamol comprising:
a. providing a pMDI comprising a container containing: (1) a carrier comprising: (i) from greater than about 90% by weight to less than 98% by weight of HFO-1234ze(E); (ii) from greater than 2% by weight to less than about 10% by weight of ethanol and (iii) from about 0.005% by weight to about 0.01% by weight of polyethylene glycol; and (2) fine particles of salbutamol suspended in one or more of said carrier components; and
b. actuating said MDI to produce an aerosol spray of said salbutamol, wherein said spray has an RSD % of not greater than 20% and a delivered dose of at least about 165 μg/actuation.

For the purposes of convenience, methods according to this paragraph are referred to as Pharmaceutical Delivery Method 4A.

The present invention also includes methods for delivering a target dose of salbutamol sulfate comprising:
a. providing a pMDI comprising a container containing: (1) a carrier comprising: (i) from greater than about 90% by weight to less than 98% by weight of HFO-1234ze(E); (ii) from greater than 2% by weight to less than about 10% by weight of ethanol and (iii) from about 0.005% by weight to about 0.01% by weight of polyethylene glycol; and (2) fine particles of salbutamol sulfate suspended in one or more of said carrier components; and
b. actuating said MDI to produce an aerosol spray of said salbutamol sulfate, wherein said spray has an RSD % of not greater than 20% and a delivered dose of at least about 165 μg/actuation.

For the purposes of convenience, methods according to this paragraph are referred to as Pharmaceutical Delivery Method 4B.

The present invention also includes methods for delivering a target dose of salbutamol comprising:
a. providing a pMDI comprising a container containing: (1) a carrier comprising: (i) from greater than about 90% by weight to less than 98% by weight of HFO-1234ze(E); (ii) from greater than 2% by weight to less than about 10% by weight of ethanol and (iii) from about 0.005% by weight to about 0.01% by weight of polyethylene glycol; and (2) fine particles of salbutamol suspended in one or more of said carrier components; and
b. actuating said MDI to produce an aerosol spray of said salbutamol, wherein said spray has an RSD % of not greater than 15% and a delivered dose of at least about 150 μg/actuation.

For the purposes of convenience, methods according to this paragraph are referred to as Pharmaceutical Delivery Method 5A.

The present invention also includes methods for delivering a target dose of salbutamol sulfate comprising:
a. providing a pMDI comprising a container containing: (1) a carrier comprising: (i) from greater than about 90% by weight to less than 98% by weight of HFO-1234ze(E); (ii) from greater than 2% by weight to less than about 10% by weight of ethanol and (iii) from about 0.005% by weight to about 0.01% by weight of polyethylene glycol; and (2) fine particles of salbutamol sulfate suspended in one or more of said carrier components; and
b. actuating said MDI to produce an aerosol spray of said salbutamol sulfate, wherein said spray has an RSD % of not greater than 15% and a delivered dose of at least about 150 μg/actuation.

For the purposes of convenience, methods according to this paragraph are referred to as Pharmaceutical Delivery Method 5B.

The present invention also includes methods for delivering a target dose of salbutamol comprising:
a. providing a pMDI comprising a container containing: (1) a carrier comprising: (i) from greater than about 90% by weight to less than 98% by weight of HFO-1234ze(E); (ii) from greater than 2% by weight to less than about 10% by weight of ethanol and (iii) from about 0.005% by weight to about 0.01% by weight of polyethylene glycol; and (2) fine particles of salbutamol suspended in one or more of said carrier components; and
c. actuating said MDI to produce an aerosol spray of said salbutamol, wherein said spray has an RSD % of not greater than 10% and a delivered dose of at least about 170 μg/actuation.

For the purposes of convenience, methods according to this paragraph are referred to as Pharmaceutical Delivery Method 6A.

The present invention also includes methods for delivering a target dose of salbutamol sulfate comprising:
a. providing a pMDI comprising a container containing: (1) a carrier comprising: (i) from greater than about 90% by weight to less than 98% by weight of HFO-1234ze(E); (ii) from greater than 2% by weight to less than about 10% by weight of ethanol and (iii) from about 0.005% by weight to about 0.01% by weight of polyethylene glycol; and (2) fine particles of salbutamol sulfate suspended in one or more of said carrier components; and
b. actuating said MDI to produce an aerosol spray of said salbutamol sulfate, wherein said spray has an RSD % of not greater than 10% and a delivered dose of at least about 170 μg/actuation.

For the purposes of convenience, methods according to this paragraph are referred to as Pharmaceutical Delivery Method 6B.

The present invention also includes methods for delivering a target dose of salbutamol comprising:
a. providing a pMDI comprising a container containing: (1) a carrier comprising: (i) from greater than about 90% by weight to less than 98% by weight of HFO-1234ze(E); (ii) from greater than 2% by weight to less than about 10% by weight of ethanol and (iii) from about 0.005% by weight to about 0.01% by weight of polyethylene glycol; and (2) fine particles of salbutamol suspended in one or more of said carrier components; and
d. actuating said MDI to produce an aerosol spray of said salbutamol, wherein said spray has an RSD % of not greater than about 5% and a delivered dose of at least about 170 μg/actuation.

For the purposes of convenience, methods according to this paragraph are referred to as Pharmaceutical Delivery Method 7A.

The present invention also includes methods for delivering a target dose of salbutamol sulfate comprising:
a. providing a pMDI comprising a container containing: (1) a carrier comprising: (i) from greater than about 90% by weight to less than 98% by weight of HFO-1234ze(E); (ii) from greater than 2% by weight to less than about 10% by weight of ethanol and (iii) from about 0.005% by weight to about 0.01% by weight of polyethylene glycol; and (2) fine particles of salbutamol sulfate suspended in one or more of said carrier components; and
b. actuating said MDI to produce an aerosol spray of said salbutamol sulfate, wherein said spray has an RSD % of not greater than about 5% and a delivered dose of at least about 170 μg/actuation.

For the purposes of convenience, methods according to this paragraph are referred to as Pharmaceutical Delivery Method 7B.

The present invention also includes methods for delivering a target dose of salbutamol comprising:
a. providing a pMDI comprising a container containing: (1) a carrier comprising: (i) from greater than about 92% by weight to less than 97% by weight of HFO-1234ze(E); (ii) from greater than 3% by weight to less than about 8% by weight of ethanol and (iii) from about 0.005% by weight to about 0.01% by weight of polyethylene glycol; and (2) fine particles of salbutamol suspended in one or more of said carrier components; and
e. actuating said MDI to produce an aerosol spray of said salbutamol, wherein said spray has an RSD % of not greater than about 5% and a delivered dose of at least about 170 μg/actuation.

For the purposes of convenience, methods according to this paragraph are referred to as Pharmaceutical Delivery Method 8A.

The present invention also includes methods for delivering a target dose of salbutamol sulfate comprising:
a. providing a pMDI comprising a container containing: ((1) a carrier comprising: (i) from greater than about 92% by weight to less than 97% by weight of HFO-1234ze(E); (ii) from greater than 3% by weight to less than about 8% by weight of ethanol and (iii) from about 0.005% by weight to about 0.01% by weight of polyethylene glycol; and (2) fine particles of salbutamol sulfate suspended in one or more of said carrier components; and
b. actuating said MDI to produce an aerosol spray of said salbutamol sulfate, wherein said spray has an RSD % of not greater than about 5% and a delivered dose of at least about 170 μg/actuation.

For the purposes of convenience, methods according to this paragraph are referred to as Pharmaceutical Delivery Method 8B.

The present invention also includes methods for delivering a target dose of salbutamol comprising:
a. providing a pMDI comprising a container containing: (1) a carrier comprising: (i) from greater than about 93% by weight to less than 97% by weight of HFO-1234ze(E); (ii) from greater than 3% by weight to less than about 7% by weight of ethanol and (iii) from about 0.005% by weight to about 0.01% by weight of polyethylene glycol; and (2) fine particles of salbutamol suspended in one or more of said carrier components; and
b. actuating said MDI to produce an aerosol spray of said salbutamol, wherein said spray has an RSD % of not greater than about 5% and a delivered dose of at least about 170 μg/actuation.

For the purposes of convenience, methods according to this paragraph are referred to as Pharmaceutical Delivery Method 9A.

The present invention also includes methods for delivering a target dose of salbutamol sulfate comprising:
a. providing a pMDI comprising a container containing: (1) a carrier comprising: (i) from greater than about 93% by weight to less than 97% by weight of HFO-1234ze(E); (ii) from greater than 3% by weight to less than about 7% by weight of ethanol; and (iii) from about 0.005% by weight to about 0.01% by weight of polyethylene glycol; and (2) fine particles of salbutamol sulfate suspended in one or more of said carrier components; and
b. actuating said MDI to produce an aerosol spray of said salbutamol sulfate, wherein said spray has an RSD % of not greater than about 5% and a delivered dose of at least about 150 μg/actuation.

For the purposes of convenience, methods according to this paragraph are referred to as Pharmaceutical Delivery Method 9B.

The present invention also includes methods for delivering a target dose of salbutamol comprising:
a. providing a pMDI comprising a container containing: (1) a carrier comprising: (i) about 95% by weight of HFO-1234ze(E); (ii) about 5% by weight of ethanol and (iii) from about 0.005% by weight to about 0.01% by weight of polyethylene glycol; and (2) fine particles of salbutamol suspended in one or more of said carrier components; and
b. actuating said MDI to produce an aerosol spray of said salbutamol, wherein said spray has an RSD % of not greater than about 5% and a delivered dose of at least about 150 μg/actuation.

For the purposes of convenience, methods according to this paragraph are referred to as Pharmaceutical Delivery Method 10A.

The present invention also includes methods for delivering a target dose of salbutamol sulfate comprising:
a. providing a pMDI comprising a container containing: (1) a carrier comprising: (i) about 95% by weight of HFO-1234ze(E); (ii) about 5% by weight of ethanol; and (iii) from about 0.005% by weight to about 0.01% by weight of polyethylene glycol; and (2) fine particles of salbutamol sulfate suspended in one or more of said carrier components; and
b. actuating said MDI to produce an aerosol spray of said salbutamol sulfate, wherein said spray has an RSD % of not greater than about 5% and a delivered dose of at least about 150 μg/actuation.

For the purposes of convenience, methods according to this paragraph are referred to as Pharmaceutical Delivery Method 10B.

The present invention also includes a pressurized metered dose inhaler (pMDI) comprising:
a. a container;
b. a pharmaceutical composition under pressure in the container, said pharmaceutical composition comprising: (i) from greater than about 90% by weight to less than 98% by weight of HFO-1234ze(E); (ii) from greater than 2% by weight to less than about 10% by weight of ethanol; (iii) from about 0.005% by weight to about 0.01% by weight of polyethylene glycol and (iv) fine particles of salbutamol suspended in said ethanol and/or said 1234ze(E); and
c. a normally closed valve on the container, said normally closed valve being actuatable to an open position to release an aerosol spray of said pharmaceutical composition from said container.

For the purposes of convenience, pMDIs according to this paragraph are referred to as pMDI 1A.

The present invention also includes a pressurized metered dose inhaler (pMDI) comprising:
a. a container:
b. a pharmaceutical composition under pressure in the container, said pharmaceutical composition comprising: (i) from greater than about 90% by weight to less than 98% by weight of HFO-1234ze(E); (ii) from greater than 2% by weight to less than about 10% by weight of ethanol; (iii) from about 0.005% by weight to about 0.01% by weight of polyethylene glycol and (iv) fine particles of salbutamol suspended in said ethanol and/or said 1234ze(E); and
d. a normally closed valve on the container, said normally closed valve being actuatable to an open position to release an aerosol spray of said pharmaceutical composition from said container.
   For the purposes of convenience, pMDIs according to this paragraph are referred to as pMDI 1B.

The present invention also includes a pressurized metered dose inhaler (pMDI) comprising:
c. a container;
d. a pharmaceutical composition under pressure in the container, said pharmaceutical composition comprising: (i) from greater than about 90% by weight to less than 98% by weight of HFO-1234ze(E); (ii) from greater than 2% by weight to less than about 10% by weight of ethanol; (iii) from about 0.005% by weight to about 0.01% by weight of polyethylene glycol and (iv) from about 2.5 mg/mL to about 3.5 mg/mL of salbutamol sulfate, said salbutamol sulfate being in the form of fine particles suspended in said ethanol and/or said 1234ze(E); and
e. a normally closed valve on the container, said normally closed valve being actuatable to an open position to release an aerosol spray of a predefined volume of said pharmaceutical composition from said container.
   For the purposes of convenience, pMDIs according to this paragraph are referred to as pMDI 2.

The present invention also includes a metered dose inhaler (MDI) comprising:
a. a container;
b. a pharmaceutical composition of the present invention, including each of Pharmaceutical Compositions 1-10, under pressure in the container; and
c. a normally closed valve on the container, said normally closed valve being actuatable to an open position to release an aerosol spray of said pharmaceutical composition from said container.

For the purposes of convenience, pMDIs according to this paragraph are referred to as pMDI 3.

### BRIEF DESCRIPTION OF THE DRAWING

The invention will now be described with reference to the accompanying drawings in which:

FIG. 1 is a cross-sectional side view of an inhaler including a canister containing a valve according to the present disclosure.

FIG. 2 is a detailed cross-sectional side view of the inhaler of FIG. 1.

FIG. 3 is a cross-sectional side view of a metering valve for an inhaler.

FIG. 4 is a chart showing the data from Example 1.

### DETAILED DESCRIPTION OF PREFERRED EMBODIMENTS

### I. Definitions

For the purposes of this invention, the term “about” in relation to the amounts expressed in weight percent for amounts greater than 2% means that the amount of the component can vary by an amount of +/−1% by weight.

For the purposes of this invention, the term “about” in relation to the amounts expressed in weight percent for amounts less than 2% and greater than 1% means that the amount of the component can vary by an amount of +/−0.2% by weight.

For the purposes of this invention, the term “about” in relation to the amounts expressed in weight percent for amounts less than 1% and greater than 0.5% means that the amount of the component can vary by an amount of +/−0.1% by weight.

For the purposes of this invention, the term “about” in relation to the amounts expressed in weight percent for amounts less than 0.01% and greater than 0.005% means that the amount of the component can vary by an amount of +/−0.001% by weight.

For the purposes of this invention, the term “about” in relation to the amounts expressed in milligrams/milliliter (mg/mL) means that the amount of the component can vary by an amount of +/−0.05 mg/mL.

For the purposes of this invention, the term “about” in relation to the amounts of a dose expressed in micrograms (μg) means that the amount of the component can vary by an amount of +/−5 μg.

For the purposes of this invention, the term “composition” is used in the broad sense to include both single phase compositions and compositions that comprise two or more phases, such as for example compositions comprising a continuous liquid phase and solid particles suspended or dispersed in the liquid phase, or for example a gaseous phase with solid particles suspended, dispersed and/or carried by the gaseous phase, as would occur in an aerosol composition.

The term “pharmaceutical composition” is used herein to include any composition which comprises at least one agent, ingredient, drug, compound, composition, or other substance that may be used on, or administered to, a human or animal for a purpose that includes one or more of therapeutic, pharmaceutical, pharmacological, diagnostic, and prophylactic and immunomodulation.

The term “fine particles” means particles that have of a mean particle diameter up to about 10 microns (10μ).

The term “delivered dose” and its abbreviation “DD” refers to the mean amount of salbutamol particles contained in the volume of composition that exits the actuator nozzle of a pMDI and is available to be drawn into a patient's lungs as determined by the procedures described in the examples hereof.

The term RSD % means the relative standard deviation of a delivered dose as measured in accordance with the examples hereof.

As used herein, the term “carrier” refers to one or more pharmacologically inert substances which provide a continuous phase in which salbutamol particles (including salbutamol sulfate particles) are suspended and which comprise components that exert a sufficiently high vapor pressure at normal room temperature to propel the particles from the canister of an MDI to a patient upon actuation of the MDI's metering valve. Therefore, the term “carrier” encompasses both a single component and a combination of two or more different components that form the medium in which the salbutamol is suspended or otherwise carried. Thus, the 1234ze(E) component of the carrier of present composition acts at least as a propellent.

The terms “HFC-134a” and “R134a” means 1,1,1,2-tetrafluoroethane.

The terms “HFO-1234ze(E),” and “1234ze(E)” as used herein each mean trans-1,3,3,3-tetrafluoropropene. Unless otherwise stated, “HFO-1234ze” and “1234ze” mean trans-1,3,3,3-tetrafluoropropene.

The term “salbutamol” as used herein encompasses any and all pharmaceutically acceptable versions of salbutamol, including pharmaceutically acceptable salts of salbutamol (such as salbutamol sulfate) and pharmaceutically acceptable esters of salbutamol.

Reference herein to a group of defined items includes all such defined items, including all such items with suffix designations. Thus, for example, a reference herein to “Pharmaceutical Compositions 1-10,” is a specific reference to each of Pharmaceutical Compositions 1A1, 1A2, 1A3, 1A4, 1A5, 1A6, 1A7, 1A8, 1A9, 1A10, 2A, 2B, 2C, 3A, 3B, 3C and so on.

### II. The Compositions

The preferred pharmaceutical compositions of the present invention, including each of Pharmaceutical Compositions 1-10 as defined above and Pharmaceutical Compositions 11-36 identified in Table 1A below, are preferably suspensions of the salbutamol in one or more of the carrier components of the composition, including particularly the HFO-1234ze(E) and/or ethanol.

The preferred pharmaceutical compositions of the present invention, including each of Pharmaceutical Compositions 1-10 as defined above and Pharmaceutical Compositions 11-36 identified in Table 1A below, are physically stable. The preferred pharmaceutical compositions of the present invention, including each of 1-10 as defined above and Pharmaceutical Compositions 11-36 identified in Table 1A below, are chemically stable. The preferred pharmaceutical compositions of the present invention, including each of Pharmaceutical Compositions 1-10 as defined above and Pharmaceutical Compositions 11-36 identified in Table 1A below, are physically stable and chemically stable.

### Concentration of Components

The concentration of the components in the present compositions can generally vary widely within the broad scope of the present invention. The concentration of the salbutamol contained in the compositions of the present invention, including each of Pharmaceutical Compositions 1-10, measured as milligrams per milliliter (mg/mL) is preferably from greater than 2.5 mg/mL, or from greater than about 2.5 mg/mL to less than about 5 mg/mL, or from greater than about 2.5 mg/mL to less than about 4.5 mg/mL, or from greater than 2.5 mg/mL to less than about 4.0 mg/mL. Preferred compositions include those identified in the following Table 1A, with the following designations in the table having the following meanings: “Comp” means that the composition comprises the identified components; CEO means that the composition consists essentially of the identified components; CO means that the composition consists of the identified components.

TABLE 1A

| Pharmaceutical | | | | | | Salbutamol |
|---|---|---|---|---|---|---|
| Composition | | Components*, wt % | | | | sulfate mg/ml |
| No. | | 1234ze(E) | Ethanol | PEG | PEG MW* | in suspension* |
| PC11A | Comp | >90-<98 | >2-<10 | 0.005 − | 300-1300 | NR |
| | | | | 0.01 | | |
| PC11B | CEO | >90-<98 | >2-<10 | 0.005 − | 300-1300 | NR |
| | | | | 0.01 | | |
| PC11C | CO | >90-<98 | >2-<10 | 0.005 − | 300-1300 | NR |
| | | | | 0.01 | | |
| PC11D | Comp | >90-<98 | >2-<10 | 0.005 − | 300-1300 | >2 |
| | | | | 0.01 | | |
| PC11E | CEO | >90-<98 | >2-<10 | 0.005 − | 300-1300 | >2 |
| | | | | 0.01 | | |
| PC11F | CO | >90-<98 | >2-<10 | 0.005 − | 300-1300 | >2 |
| | | | | 0.01 | | |
| PC11G | Comp | >90-<98 | >2-<10 | 0.005 − | 300-1300 | >2.5-<5 |
| | | | | 0.01 | | |
| PC11H | CEO | >90-<98 | >2-<10 | 0.005 − | 300-1300 | >2.5-<5 |
| | | | | 0.01 | | |
| PC11I | CO | >90-<98 | >2-<10 | 0.005 − | 300-1300 | >2.5-<5 |
| | | | | 0.01 | | |
| PC11J | Comp | >90-<98 | >2-<10 | 0.005 − | 300-1300 | >2.5-<4.5 |
| | | | | 0.01 | | |
| PC11K | CEO | >90-<98 | >2-<10 | 0.005 − | 300-1300 | >2.5-<4.5 |
| | | | | 0.01 | | |
| PC11L | CO | >90-<98 | >2-<10 | 0.005 − | 300-1300 | >2.5-<4.5 |
| | | | | 0.01 | | |
| PC11M | Comp | >90-<98 | >2-<10 | 0.005 − | 300-1300 | >2.5-<4 |
| | | | | 0.01 | | |
| PC11N | CEO | >90-<98 | >2-<10 | 0.005 − | 300-1300 | >2.5-<4 |
| | | | | 0.01 | | |
| PC11O | CO | >90-<98 | >2-<10 | 0.005 − | 300-1300 | >2.5-<4 |
| | | | | 0.01 | | |
| PC12A | Comp | >90-<98 | >2-<10 | 0.005 − | 400-1300 | NR |
| | | | | 0.01 | | |
| PC12B | CEO | >90-<98 | >2-<10 | 0.005 − | 400-1300 | NR |
| | | | | 0.01 | | |
| PC12C | CO | >90-<98 | >2-<10 | 0.005 − | 400-1300 | NR |
| | | | | 0.01 | | |
| PC12D | Comp | >90-<98 | >2-<10 | 0.005 − | 400-1300 | >2 |
| | | | | 0.01 | | |
| PC12E | CEO | >90-<98 | >2-<10 | 0.005 − | 400-1300 | >2 |
| | | | | 0.01 | | |
| PC12F | CO | >90-<98 | >2-<10 | 0.005 − | 400-1300 | >2 |
| | | | | 0.01 | | |
| PC12G | Comp | >90-<98 | >2-<10 | 0.005 − | 400-1300 | >2.5-<5 |
| | | | | 0.01 | | |
| PC12H | CEO | >90-<98 | >2-<10 | 0.005 − | 400-1300 | >2.5-<5 |
| | | | | 0.01 | | |
| PC12I | CC | >90-<98 | >2-<10 | 0.005 − | 400-1300 | >2.5-<5 |
| | | | | 0.01 | | |
| PC12J | Comp | >90-<98 | >2-<10 | 0.005 − | 400-1300 | >2.5-<4.5 |
| | | | | 0.01 | | |
| PC12K | CEO | >90-<98 | >2-<10 | 0.005 − | 400-1300 | >2.5-<4.5 |
| | | | | 0.01 | | |
| PC12L | CO | >90-<98 | >2-<10 | 0.005 − | 400-1300 | >2.5-<4.5 |
| | | | | 0.01 | | |
| PC12M | Comp | >90-<98 | >2-<10 | 0.005 − | 400-1300 | >2.5-<4 |
| | | | | 0.01 | | |
| PC12N | CEO | >90-<98 | >2-<10 | 0.005 − | 400-1300 | >2.5-<4 |
| | | | | 0.01 | | |
| PC12O | CO | >90-<98 | >2-<10 | 0.005 − | 400-1300 | >2.5-<4 |
| | | | | 0.01 | | |
| PC13A | Comp | >90-<98 | >2-<10 | 0.005 − | 500-1300 | NR |
| | | | | 0.01 | | |
| PC13B | CEO | >90-<98 | >2-<10 | 0.005 − | 500-1300 | NR |
| | | | | 0.01 | | |
| PC13C | CC | >90-<98 | >2-<10 | 0.005 − | 500-1300 | NR |
| | | | | 0.01 | | |
| PC13D | Comp | >90-<98 | >2-<10 | 0.005 − | 500-1300 | >2 |
| | | | | 0.01 | | |
| PC13E | CEO | >90-<98 | >2-<10 | 0.005 − | 500-1300 | >2 |
| | | | | 0.01 | | |
| PC13F | CO | >90-<98 | >2-<10 | 0.005 − | 500-1300 | >2 |
| | | | | 0.01 | | |
| PC13G | Comp | >90-<98 | >2-<10 | 0.005 − | 500-1300 | >2.5-<5 |
| | | | | 0.01 | | |
| PC13H | CEO | >90-<98 | >2-<10 | 0.005 − | 500-1300 | >2.5-<5 |
| | | | | 0.01 | | |
| PC13I | CO | >90-<98 | >2-<10 | 0.005 − | 500-1300 | >2.5-<5 |
| | | | | 0.01 | | |
| PC13J | Comp | >90-<98 | >2-<10 | 0.005 − | 500-1300 | >2.5-<4.5 |
| | | | | 0.01 | | |
| PC13K | CEO | >90-<98 | >2-<10 | 0.005 − | 500-1300 | >2.5-<4.5 |
| | | | | 0.01 | | |
| PC13L | CO | >90-<98 | >2-<10 | 0.005 − | 500-1300 | >2.5-<4.5 |
| | | | | 0.01 | | |
| PC13M | Comp | >90-<98 | >2-<10 | 0.005 − | 500-1300 | >2.5-<4 |
| | | | | 0.01 | | |
| PC13N | CEO | >90-<98 | >2-<10 | 0.005 − | 500-1300 | >2.5-<4 |
| | | | | 0.01 | | |
| PC13O | CO | >90-<98 | >2-<10 | 0.005 − | 600-1300 | >2.5-<4 |
| | | | | 0.01 | | |
| PC14A | Comp | >90-<98 | >2-<10 | 0.005 − | 600-1300 | NR |
| | | | | 0.01 | | |
| PC14B | CEO | >90-<98 | >2-<10 | 0.005 − | 600-1300 | NR |
| | | | | 0.01 | | |
| PC14C | CO | >90-<98 | >2-<10 | 0.005 − | 600-1300 | NR |
| | | | | 0.01 | | |
| PC14D | Comp | >90-<98 | >2-<10 | 0.005 − | 600-1300 | >2 |
| | | | | 0.01 | | |
| PC14E | CEO | >90-<98 | >2-<10 | 0.005 − | 600-1300 | >2 |
| | | | | 0.01 | | |
| PC14F | CO | >90-<98 | >2-<10 | 0.005 − | 600-1300 | >2 |
| | | | | 0.01 | | |
| PC14G | Comp | >90-<98 | >2-<10 | 0.005 − | 600-1300 | >2.5-<5 |
| | | | | 0.01 | | |
| PC14H | CEO | >90-<98 | >2-<10 | 0.005 − | 600-1300 | >2.5-<5 |
| | | | | 0.01 | | |
| PC14I | CO | >90-<98 | >2-<10 | 0.005 − | 600-1300 | >2.5-<5 |
| | | | | 0.01 | | |
| PC14J | Comp | >90-<98 | >2-<10 | 0.005 − | 600-1300 | >2.5-<4.5 |
| | | | | 0.01 | | |
| PC14K | CEO | >90-<98 | >2-<10 | 0.005 − | 600-1300 | >2.5-<4.5 |
| | | | | 0.01 | | |
| PC14L | CO | >90-<98 | >2-<10 | 0.005 − | 600-1300 | >2.5-<4.5 |
| | | | | 0.01 | | |
| PC14M | Comp | >90-<98 | >2-<10 | 0.005 − | 600-1300 | >2.5-<4 |
| | | | | 0.01 | | |
| PC14N | CEO | >90-<98 | >2-<10 | 0.005 − | 600-1300 | >2.5-<4 |
| | | | | 0.01 | | |
| PC14O | CO | >90-<98 | >2-<10 | 0.005 − | 600-1300 | >2.5-<4 |
| | | | | 0.01 | | |
| PC15A | Comp | >90-<98 | >2-<10 | 0.005 − | 800-1200 | NR |
| | | | | 0.01 | | |
| PC15B | CEO | >90-<98 | >2-<10 | 0.005 − | 800-1200 | NR |
| | | | | 0.01 | | |
| PC15C | CO | >90-<98 | >2-<10 | 0.005 − | 800-1200 | NR |
| | | | | 0.01 | | |
| PC15D | Comp | >90-<98 | >2-<10 | 0.005 − | 800-1200 | >2 |
| | | | | 0.01 | | |
| PC15E | CEO | >90-<98 | >2-<10 | 0.005 − | 800-1200 | >2 |
| | | | | 0.01 | | |
| PC15F | CO | >90-<98 | >2-<10 | 0.005 − | 800-1200 | >2 |
| | | | | 0.01 | | |
| PC15G | Comp | >90-<98 | >2-<10 | 0.005 − | 800-1200 | >2.5-<5 |
| | | | | 0.01 | | |
| PC15H | CEO | >90-<98 | >2-<10 | 0.005 − | 800-1200 | >2.5-<5 |
| | | | | 0.01 | | |
| PC15I | CO | >90-<98 | >2-<10 | 0.005 − | 800-1200 | >2.5-<5 |
| | | | | 0.01 | | |
| PC15J | Comp | >90-<98 | >2-<10 | 0.005 − | 800-1200 | >2.5-<4.5 |
| | | | | 0.01 | | |
| PC15K | CEO | >90-<98 | >2-<10 | 0.005 − | 800-1200 | >2.5-<4.5 |
| | | | | 0.01 | | |
| PC15L | CO | >90-<98 | >2-<10 | 0.005 − | 800-1200 | >2.5-<4.5 |
| | | | | 0.01 | | |
| PC15M | Comp | >90-<98 | >2-<10 | 0.005 − | 800-1200 | >2.5-<4 |
| | | | | 0.01 | | |
| PC15N | CEO | >90-<98 | >2-<10 | 0.005 − | 800-1200 | >2.5-<4 |
| | | | | 0.01 | | |
| PC15O | CO | >90-<98 | >2-<10 | 0.005 − | 800-1200 | >2.5-<4 |
| | | | | 0.01 | | |
| PC16A | Comp | >91-<97 | >3-<9 | 0.005 − | 300-1300 | NR |
| | | | | 0.01 | | |
| PC16B | CEO | >91-<97 | >3-<9 | 0.005 − | 300-1300 | NR |
| | | | | 0.01 | | |
| PC16C | CO | >91-<97 | >3-<9 | 0.005 − | 300-1300 | NR |
| | | | | 0.01 | | |
| PC16D | Comp | >91-<97 | >3-<9 | 0.005 − | 300-1300 | >2 |
| | | | | 0.01 | | |
| PC16E | CEO | >91-<97 | >3-<9 | 0.005 − | 300-1300 | >2 |
| | | | | 0.01 | | |
| PC16F | CO | >91-<97 | >3-<9 | 0.005 − | 300-1300 | >2 |
| | | | | 0.01 | | |
| PC16G | Comp | >91-<97 | >3-<9 | 0.005 − | 300-1300 | >2.5-<5 |
| | | | | 0.01 | | |
| PC16H | CEO | >91-<97 | >3-<9 | 0.005 − | 300-1300 | >2.5-<5 |
| | | | | 0.01 | | |
| PC16I | CO | >91-<97 | >3-<9 | 0.005 − | 300-1300 | >2.5-<5 |
| | | | | 0.01 | | |
| PC16J | Comp | >91-<97 | >3-<9 | 0.005 − | 300-1300 | >2.5-<4.5 |
| | | | | 0.01 | | |
| PC16K | CEO | >91-<97 | >3-<9 | 0.005 − | 300-1300 | >2.5-<4.5 |
| | | | | 0.01 | | |
| PC16L | CO | >91-<97 | >3-<9 | 0.005 − | 300-1300 | >2.5-<4.5 |
| | | | | 0.01 | | |
| PC16M | Comp | >91-<97 | >3-<9 | 0.005 − | 300-1300 | >2.5-<4 |
| | | | | 0.01 | | |
| PC16N | CEO | >91-<97 | >3-<9 | 0.005 − | 300-1300 | >2.5-<4 |
| | | | | 0.01 | | |
| PC16O | CO | >91-<97 | >3-<9 | 0.005 − | 300-1300 | >2.5-<4 |
| | | | | 0.01 | | |
| PC17A | Comp | >91-<97 | >3-<9 | 0.005 − | 400-1300 | NR |
| | | | | 0.01 | | |
| PC17B | CEO | >91-<97 | >3-<9 | 0.005 − | 400-1300 | NR |
| | | | | 0.01 | | |
| PC17C | CO | >91-<97 | >3-<9 | 0.005 − | 400-1300 | NR |
| | | | | 0.01 | | |
| PC17D | Comp | >91-<97 | >3-<9 | 0.005 − | 400-1300 | >2 |
| | | | | 0.01 | | |
| PC17E | CEO | >91-<97 | >3-<9 | 0.005 − | 400-1300 | >2 |
| | | | | 0.01 | | |
| PC17F | CO | >91-<97 | >3-<9 | 0.005 − | 400-1300 | >2 |
| | | | | 0.01 | | |
| PC17G | Comp | >91-<97 | >3-<9 | 0.005 − | 400-1300 | >2.5-<5 |
| | | | | 0.01 | | |
| PC17H | CEO | >91-<97 | >3-<9 | 0.005 − | 400-1300 | >2.5-<5 |
| | | | | 0.01 | | |
| PC17I | CO | >91-<97 | >3-<9 | 0.005 − | 400-1300 | >2.5-<5 |
| | | | | 0.01 | | |
| PC17J | Comp | >91-<97 | >3-<9 | 0.005 − | 400-1300 | >2.5-<4.5 |
| | | | | 0.01 | | |
| PC17K | CEO | >91-<97 | >3-<9 | 0.005 − | 400-1300 | >2.5-<4.5 |
| | | | | 0.01 | | |
| PC17L | CO | >91-<97 | >3-<9 | 0.005 − | 400-1300 | >2.5-<4.5 |
| | | | | 0.01 | | |
| PC17M | Comp | >91-<97 | >3-<9 | 0.005 − | 400-1300 | >2.5-<4 |
| | | | | 0.01 | | |
| PC17N | CEO | >91-<97 | >3-<9 | 0.005 − | 400-1300 | >2.5-<4 |
| | | | | 0.01 | | |
| PC17O | CO | >91-<97 | >3-<9 | 0.005 − | 400-1300 | >2.5-<4 |
| | | | | 0.01 | | |
| PC18A | Comp | >91-<97 | >3-<9 | 0.005 − | 500-1300 | NR |
| | | | | 0.01 | | |
| PC18B | CEO | >91-<97 | >3-<9 | 0.005 − | 500-1300 | NR |
| | | | | 0.01 | | |
| PC18C | CO | >91-<97 | >3-<9 | 0.005 − | 500-1300 | NR |
| | | | | 0.01 | | |
| PC18D | Comp | >91-<97 | >3-<9 | 0.005 − | 500-1300 | >2 |
| | | | | 0.01 | | |
| PC18E | CEO | >91-<97 | >3-<9 | 0.005 − | 500-1300 | >2 |
| | | | | 0.01 | | |
| PC18F | CO | >91-<97 | >3-<9 | 0.005 − | 500-1300 | >2 |
| | | | | 0.01 | | |
| PC18G | Comp | >91-<97 | >3-<9 | 0.005 − | 500-1300 | >2.5-<5 |
| | | | | 0.01 | | |
| PC18H | CEO | >91-<97 | >3-<9 | 0.005 − | 500-1300 | >2.5-<5 |
| | | | | 0.01 | | |
| PC18I | CC | >91-<97 | >3-<9 | 0.005 − | 500-1300 | >2.5-<5 |
| | | | | 0.01 | | |
| PC18J | Comp | >91-<97 | >3-<9 | 0.005 − | 500-1300 | >2.5-<4.5 |
| | | | | 0.01 | | |
| PC18K | CEO | >91-<97 | >3-<9 | 0.005 − | 500-1300 | >2.5-<4.5 |
| | | | | 0.01 | | |
| PC18L | CO | >91-<97 | >3-<9 | 0.005 − | 500-1300 | >2.5-<4.5 |
| | | | | 0.01 | | |
| PC18M | Comp | >91-<97 | >3-<9 | 0.005 − | 500-1300 | >2.5-<4 |
| | | | | 0.01 | | |
| PC18N | CEO | >91-<97 | >3-<9 | 0.005 − | 500-1300 | >2.5-<4 |
| | | | | 0.01 | | |
| PC18O | CO | >91-<97 | >3-<9 | 0.005 − | 600-1300 | >2.5-<4 |
| | | | | 0.01 | | |
| PC19A | Comp | >91-<97 | >3-<9 | 0.005 − | 600-1300 | NR |
| | | | | 0.01 | | |
| PC19B | CEO | >91-<97 | >3-<9 | 0.005 − | 600-1300 | NR |
| | | | | 0.01 | | |
| PC19C | CO | >91-<97 | >3-<9 | 0.005 − | 600-1300 | NR |
| | | | | 0.01 | | |
| PC19D | Comp | >91-<97 | >3-<9 | 0.005 − | 600-1300 | >2 |
| | | | | 0.01 | | |
| PC19E | CEO | >91-<97 | >3-<9 | 0.005 − | 600-1300 | >2 |
| | | | | 0.01 | | |
| PC19F | CO | >91-<97 | >3-<9 | 0.005 − | 600-1300 | >2 |
| | | | | 0.01 | | |
| PC19G | Comp | >91-<97 | >3-<9 | 0.005 − | 600-1300 | >2.5-<5 |
| | | | | 0.01 | | |
| PC19H | CEO | >91-<97 | >3-<9 | 0.005 − | 600-1300 | >2.5-<5 |
| | | | | 0.01 | | |
| PC19I | CO | >91-<97 | >3-<9 | 0.005 − | 600-1300 | >2.5-<5 |
| | | | | 0.01 | | |
| PC19J | Comp | >91-<97 | >3-<9 | 0.005 − | 600-1300 | >2.5-<4.5 |
| | | | | 0.01 | | |
| PC19K | CEO | >91-<97 | >3-<9 | 0.005 − | 600-1300 | >2.5-<4.5 |
| | | | | 0.01 | | |
| PC19L | CO | >91-<97 | >3-<9 | 0.005 − | 600-1300 | >2.5-<4.5 |
| | | | | 0.01 | | |
| PC19M | Comp | >91-<97 | >3-<9 | 0.005 − | 600-1300 | >2.5-<4 |
| | | | | 0.01 | | |
| PC19N | CEO | >91-<97 | >3-<9 | 0.005 − | 600-1300 | >2.5-<4 |
| | | | | 0.01 | | |
| PC19O | CO | >91-<97 | >3-<9 | 0.005 − | 600-1300 | >2.5-<4 |
| | | | | 0.01 | | |
| PC20A | Comp | >91-<97 | >3-<9 | 0.005 − | 800-1200 | NR |
| | | | | 0.01 | | |
| PC20B | CEO | >91-<97 | >3-<9 | 0.005 − | 800-1200 | NR |
| | | | | 0.01 | | |
| PC20C | CO | >91-<97 | >3-<9 | 0.005 − | 800-1200 | NR |
| | | | | 0.01 | | |
| PC20D | Comp | >91-<97 | >3-<9 | 0.005 − | 800-1200 | >2 |
| | | | | 0.01 | | |
| PC20E | CEO | >91-<97 | >3-<9 | 0.005 − | 800-1200 | >2 |
| | | | | 0.01 | | |
| PC20F | CO | >91-<97 | >3-<9 | 0.005 − | 800-1200 | >2 |
| | | | | 0.01 | | |
| PC20G | Comp | >91-<97 | >3-<9 | 0.005 − | 800-1200 | >2.5-<5 |
| | | | | 0.01 | | |
| PC20H | CEO | >91-<97 | >3-<9 | 0.005 − | 800-1200 | >2.5-<5 |
| | | | | 0.01 | | |
| PC20I | CO | >91-<97 | >3-<9 | 0.005 − | 800-1200 | >2.5-<5 |
| | | | | 0.01 | | |
| PC20J | Comp | >91-<97 | >3-<9 | 0.005 − | 800-1200 | >2.5-<4.5 |
| | | | | 0.01 | | |
| PC20K | CEO | >91-<97 | >3-<9 | 0.005 − | 800-1200 | >2.5-<4.5 |
| | | | | 0.01 | | |
| PC20L | CO | >91-<97 | >3-<9 | 0.005 − | 800-1200 | >2.5-<4.5 |
| | | | | 0.01 | | |
| PC20M | Comp | >91-<97 | >3-<9 | 0.005 − | 800-1200 | >2.5-<4 |
| | | | | 0.01 | | |
| PC20N | CEO | >91-<97 | >3-<9 | 0.005 − | 800-1200 | >2.5-<4 |
| | | | | 0.01 | | |
| PC20O | CO | >91-<97 | >3-<9 | 0.005 − | 800-1200 | >2.5-<4 |
| | | | | 0.01 | | |
| PC21A | Comp | >92-<97 | >3-<8 | 0.005 − | 300-1300 | NR |
| | | | | 0.01 | | |
| PC21B | CEO | >92-<97 | >3-<8 | 0.005 − | 300-1300 | NR |
| | | | | 0.01 | | |
| PC21C | CO | >92-<97 | >3-<8 | 0.005 − | 300-1300 | NR |
| | | | | 0.01 | | |
| PC21D | Comp | >92-<97 | >3-<8 | 0.005 − | 300-1300 | >2 |
| | | | | 0.01 | | |
| PC21E | CEO | >92-<97 | >3-<8 | 0.005 − | 300-1300 | >2 |
| | | | | 0.01 | | |
| PC21F | CO | >92-<97 | >3-<8 | 0.005 − | 300-1300 | >2 |
| | | | | 0.01 | | |
| PC21G | Comp | >92-<97 | >3-<8 | 0.005 − | 300-1300 | >2.5-<5 |
| | | | | 0.01 | | |
| PC21H | CEO | >92-<97 | >3-<8 | 0.005 − | 300-1300 | >2.5-<5 |
| | | | | 0.01 | | |
| PC21I | CO | >92-<97 | >3-<8 | 0.005 − | 300-1300 | >2.5-<5 |
| | | | | 0.01 | | |
| PC21J | Comp | >92-<97 | >3-<8 | 0.005 − | 300-1300 | >2.5-<4.5 |
| | | | | 0.01 | | |
| PC21K | CEO | >92-<97 | >3-<8 | 0.005 − | 300-1300 | >2.5-<4.5 |
| | | | | 0.01 | | |
| PC21L | CO | >92-<97 | >3-<8 | 0.005 − | 300-1300 | >2.5-<4.5 |
| | | | | 0.01 | | |
| PC21M | Comp | >92-<97 | >3-<8 | 0.005 − | 300-1300 | >2.5-<4 |
| | | | | 0.01 | | |
| PC21N | CEO | >92-<97 | >3-<8 | 0.005 − | 300-1300 | >2.5-<4 |
| | | | | 0.01 | | |
| PC21O | CO | >92-<97 | >3-<8 | 0.005 − | 300-1300 | >2.5-<4 |
| | | | | 0.01 | | |
| PC22A | Comp | >92-<97 | >3-<8 | 0.005 − | 400-1300 | NR |
| | | | | 0.01 | | |
| PC22B | CEO | >92-<97 | >3-<8 | 0.005 − | 400-1300 | NR |
| | | | | 0.01 | | |
| PC22C | CO | >92-<97 | >3-<8 | 0.005 − | 400-1300 | NR |
| | | | | 0.01 | | |
| PC22D | Comp | >92-<97 | >3-<8 | 0.005 − | 400-1300 | >2 |
| | | | | 0.01 | | |
| PC22E | CEO | >92-<97 | >3-<8 | 0.005 − | 400-1300 | >2 |
| | | | | 0.01 | | |
| PC22F | CO | >92-<97 | >3-<8 | 0.005 − | 400-1300 | >2 |
| | | | | 0.01 | | |
| PC22G | Comp | >92-<97 | >3-<8 | 0.005 − | 400-1300 | >2.5-<5 |
| | | | | 0.01 | | |
| PC22H | CEO | >92-<97 | >3-<8 | 0.005 − | 400-1300 | >2.5-<5 |
| | | | | 0.01 | | |
| PC22I | CO | >92-<97 | >3-<8 | 0.005 − | 400-1300 | >2.5-<5 |
| | | | | 0.01 | | |
| PC22J | Comp | >92-<97 | >3-<8 | 0.005 − | 400-1300 | >2.5-<4.5 |
| | | | | 0.01 | | |
| PC22K | CEO | >92-<97 | >3-<8 | 0.005 − | 400-1300 | >2.5-<4.5 |
| | | | | 0.01 | | |
| PC22L | CO | >92-<97 | >3-<8 | 0.005 − | 400-1300 | >2.5-<4.5 |
| | | | | 0.01 | | |
| PC22M | Comp | >92-<97 | >3-<8 | 0.005 − | 400-1300 | >2.5-<4 |
| | | | | 0.01 | | |
| PC22N | CEO | >92-<97 | >3-<8 | 0.005 − | 400-1300 | >2.5-<4 |
| | | | | 0.01 | | |
| PC22O | CO | >92-<97 | >3-<8 | 0.005 − | 400-1300 | >2.5-<4 |
| | | | | 0.01 | | |
| PC23A | Comp | >92-<97 | >3-<8 | 0.005 − | 500-1300 | NR |
| | | | | 0.01 | | |
| PC23B | CEO | >92-<97 | >3-<8 | 0.005 − | 500-1300 | NR |
| | | | | 0.01 | | |
| PC23C | CO | >92-<97 | >3-<8 | 0.005 − | 500-1300 | NR |
| | | | | 0.01 | | |
| PC23D | Comp | >92-<97 | >3-<8 | 0.005 − | 500-1300 | >2 |
| | | | | 0.01 | | |
| PC23E | CEO | >92-<97 | >3-<8 | 0.005 − | 500-1300 | >2 |
| | | | | 0.01 | | |
| PC23F | CO | >92-<97 | >3-<8 | 0.005 − | 500-1300 | >2 |
| | | | | 0.01 | | |
| PC23G | Comp | >92-<97 | >3-<8 | 0.005 − | 500-1300 | >2.5-<5 |
| | | | | 0.01 | | |
| PC23H | CEO | >92-<97 | >3-<8 | 0.005 − | 500-1300 | >2.5-<5 |
| | | | | 0.01 | | |
| PC23I | CC | >92-<97 | >3-<8 | 0.005 − | 500-1300 | >2.5-<5 |
| | | | | 0.01 | | |
| PC23J | Comp | >92-<97 | >3-<8 | 0.005 − | 500-1300 | >2.5-<4.5 |
| | | | | 0.01 | | |
| PC23K | CEO | >92-<97 | >3-<8 | 0.005 − | 500-1300 | >2.5-<4.5 |
| | | | | 0.01 | | |
| PC23L | CC | >92-<97 | >3-<8 | 0.005 − | 500-1300 | >2.5-<4.5 |
| | | | | 0.01 | | |
| PC23M | Comp | >92-<97 | >3-<8 | 0.005 − | 500-1300 | >2.5-<4 |
| | | | | 0.01 | | |
| PC23N | CEO | >92-<97 | >3-<8 | 0.005 − | 500-1300 | >2.5-<4 |
| | | | | 0.01 | | |
| PC23O | CO | >92-<97 | >3-<8 | 0.005 − | 600-1300 | >2.5-<4 |
| | | | | 0.01 | | |
| PC24A | Comp | >92-<97 | >3-<8 | 0.005 − | 600-1300 | NR |
| | | | | 0.01 | | |
| PC24B | CEO | >92-<97 | >3-<8 | 0.005 − | 600-1300 | NR |
| | | | | 0.01 | | |
| PC24C | CO | >92-<97 | >3-<8 | 0.005 − | 600-1300 | NR |
| | | | | 0.01 | | |
| PC24D | Comp | >92-<97 | >3-<8 | 0.005 − | 600-1300 | >2 |
| | | | | 0.01 | | |
| PC24E | CEO | >92-<97 | >3-<8 | 0.005 − | 600-1300 | >2 |
| | | | | 0.01 | | |
| PC24F | CC | >92-<97 | >3-<8 | 0.005 − | 600-1300 | >2 |
| | | | | 0.01 | | |
| PC24G | Comp | >92-<97 | >3-<8 | 0.005 − | 600-1300 | >2.5-<5 |
| | | | | 0.01 | | |
| PC24H | CEO | >92-<97 | >3-<8 | 0.005 − | 600-1300 | >2.5-<5 |
| | | | | 0.01 | | |
| PC24I | CC | >92-<97 | >3-<8 | 0.005 − | 600-1300 | >2.5-<5 |
| | | | | 0.01 | | |
| PC24J | Comp | >92-<97 | >3-<8 | 0.005 − | 600-1300 | >2.5-<4.5 |
| | | | | 0.01 | | |
| PC24K | CEO | >92-<97 | >3-<8 | 0.005 − | 600-1300 | >2.5-<4.5 |
| | | | | 0.01 | | |
| PC24L | CO | >92-<97 | >3-<8 | 0.005 − | 600-1300 | >2.5-<4.5 |
| | | | | 0.01 | | |
| PC24M | Comp | >92-<97 | >3-<8 | 0.005 − | 600-1300 | >2.5-<4 |
| | | | | 0.01 | | |
| PC24N | CEO | >92-<97 | >3-<8 | 0.005 − | 600-1300 | >2.5-<4 |
| | | | | 0.01 | | |
| PC24O | CO | >92-<97 | >3-<8 | 0.005 − | 600-1300 | >2.5-<4 |
| | | | | 0.01 | | |
| PC25A | Comp | >92-<97 | >3-<8 | 0.005 − | 800-1200 | NR |
| | | | | 0.01 | | |
| PC25B | CEO | >92-<97 | >3-<8 | 0.005 − | 800-1200 | NR |
| | | | | 0.01 | | |
| PC25C | CO | >92-<97 | >3-<8 | 0.005 − | 800-1200 | NR |
| | | | | 0.01 | | |
| PC25D | Comp | >92-<97 | >3-<8 | 0.005 − | 800-1200 | >2 |
| | | | | 0.01 | | |
| PC25E | CEO | >92-<97 | >3-<8 | 0.005 − | 800-1200 | >2 |
| | | | | 0.01 | | |
| PC25F | CO | >92-<97 | >3-<8 | 0.005 − | 800-1200 | >2 |
| | | | | 0.01 | | |
| PC25G | Comp | >92-<97 | >3-<8 | 0.005 − | 800-1200 | >2.5-<5 |
| | | | | 0.01 | | |
| PC25H | CEO | >92-<97 | >3-<8 | 0.005 − | 800-1200 | >2.5-<5 |
| | | | | 0.01 | | |
| PC25I | CO | >92-<97 | >3-<8 | 0.005 − | 800-1200 | >2.5-<5 |
| | | | | 0.01 | | |
| PC25J | Comp | >92-<97 | >3-<8 | 0.005 − | 800-1200 | >2.5-<4.5 |
| | | | | 0.01 | | |
| PC25K | CEO | >92-<97 | >3-<8 | 0.005 − | 800-1200 | >2.5-<4.5 |
| | | | | 0.01 | | |
| PC25L | CO | >92-<97 | >3-<8 | 0.005 − | 800-1200 | >2.5-<4.5 |
| | | | | 0.01 | | |
| PC25M | Comp | >92-<97 | >3-<8 | 0.005 − | 800-1200 | >2.5-<4 |
| | | | | 0.01 | | |
| PC25N | CEO | >92-<97 | >3-<8 | 0.005 − | 800-1200 | >2.5-<4 |
| | | | | 0.01 | | |
| PC25O | CO | >92-<97 | >3-<8 | 0.005 − | 800-1200 | >2.5-<4 |
| | | | | 0.01 | | |
| PC26A | Comp | >93-<97 | >3-<7 | 0.005 − | 300-1300 | NR |
| | | | | 0.01 | | |
| PC26B | CEO | >93-<97 | >3-<7 | 0.005 − | 300-1300 | NR |
| | | | | 0.01 | | |
| PC26C | CO | >93-<97 | >3-<7 | 0.005 − | 300-1300 | NR |
| | | | | 0.01 | | |
| PC26D | Comp | >93-<97 | >3-<7 | 0.005 − | 300-1300 | >2 |
| | | | | 0.01 | | |
| PC26E | CEO | >93-<97 | >3-<7 | 0.005 − | 300-1300 | >2 |
| | | | | 0.01 | | |
| PC26F | CO | >93-<97 | >3-<7 | 0.005 − | 300-1300 | >2 |
| | | | | 0.01 | | |
| PC26G | Comp | >93-<97 | >3-<7 | 0.005 − | 300-1300 | >2.5-<5 |
| | | | | 0.01 | | |
| PC26H | CEO | >93-<97 | >3-<7 | 0.005 − | 300-1300 | >2.5-<5 |
| | | | | 0.01 | | |
| PC26I | CO | >93-<97 | >3-<7 | 0.005 − | 300-1300 | >2.5-<5 |
| | | | | 0.01 | | |
| PC26J | Comp | >93-<97 | >3-<7 | 0.005 − | 300-1300 | >2.5-<4.5 |
| | | | | 0.01 | | |
| PC26K | CEO | >93-<97 | >3-<7 | 0.005 − | 300-1300 | >2.5-<4.5 |
| | | | | 0.01 | | |
| PC26L | CO | >93-<97 | >3-<7 | 0.005 − | 300-1300 | >2.5-<4.5 |
| | | | | 0.01 | | |
| PC26M | Comp | >93-<97 | >3-<7 | 0.005 − | 300-1300 | >2.5-<4 |
| | | | | 0.01 | | |
| PC26N | CEO | >93-<97 | >3-<7 | 0.005 − | 300-1300 | >2.5-<4 |
| | | | | 0.01 | | |
| PC26O | CO | >93-<97 | >3-<7 | 0.005 − | 300-1300 | >2.5-<4 |
| | | | | 0.01 | | |
| PC27A | Comp | >93-<97 | >3-<7 | 0.005 − | 400-1300 | NR |
| | | | | 0.01 | | |
| PC27B | CEO | >93-<97 | >3-<7 | 0.005 − | 400-1300 | NR |
| | | | | 0.01 | | |
| PC27C | CO | >93-<97 | >3-<7 | 0.005 − | 400-1300 | NR |
| | | | | 0.01 | | |
| PC27D | Comp | >93-<97 | >3-<7 | 0.005 − | 400-1300 | >2 |
| | | | | 0.01 | | |
| PC27E | CEO | >93-<97 | >3-<7 | 0.005 − | 400-1300 | >2 |
| | | | | 0.01 | | |
| PC27F | CO | >93-<97 | >3-<7 | 0.005 − | 400-1300 | >2 |
| | | | | 0.01 | | |
| PC27G | Comp | >93-<97 | >3-<7 | 0.005 − | 400-1300 | >2.5-<5 |
| | | | | 0.01 | | |
| PC27H | CEO | >93-<97 | >3-<7 | 0.005 − | 400-1300 | >2.5-<5 |
| | | | | 0.01 | | |
| PC27I | CO | >93-<97 | >3-<7 | 0.005 − | 400-1300 | >2.5-<5 |
| | | | | 0.01 | | |
| PC27J | Comp | >93-<97 | >3-<7 | 0.005 − | 400-1300 | >2.5-<4.5 |
| | | | | 0.01 | | |
| PC27K | CEO | >93-<97 | >3-<7 | 0.005 − | 400-1300 | >2.5-<4.5 |
| | | | | 0.01 | | |
| PC27L | CO | >93-<97 | >3-<7 | 0.005 − | 400-1300 | >2.5-<4.5 |
| | | | | 0.01 | | |
| PC27M | Comp | >93-<97 | >3-<7 | 0.005 − | 400-1300 | >2.5-<4 |
| | | | | 0.01 | | |
| PC27N | CEO | >93-<97 | >3-<7 | 0.005 − | 400-1300 | >2.5-<4 |
| | | | | 0.01 | | |
| PC27O | CO | >93-<97 | >3-<7 | 0.005 − | 400-1300 | >2.5-<4 |
| | | | | 0.01 | | |
| PC28A | Comp | >93-<97 | >3-<7 | 0.005 − | 500-1300 | NR |
| | | | | 0.01 | | |
| PC28B | CEO | >93-<97 | >3-<7 | 0.005 − | 500-1300 | NR |
| | | | | 0.01 | | |
| PC28C | CO | >93-<97 | >3-<7 | 0.005 − | 500-1300 | NR |
| | | | | 0.01 | | |
| PC28D | Comp | >93-<97 | >3-<7 | 0.005 − | 500-1300 | >2 |
| | | | | 0.01 | | |
| PC28E | CEO | >93-<97 | >3-<7 | 0.005 − | 500-1300 | >2 |
| | | | | 0.01 | | |
| PC28F | CO | >93-<97 | >3-<7 | 0.005 − | 500-1300 | >2 |
| | | | | 0.01 | | |
| PC28G | Comp | >93-<97 | >3-<7 | 0.005 − | 500-1300 | >2.5-<5 |
| | | | | 0.01 | | |
| PC28H | CEO | >93-<97 | >3-<7 | 0.005 − | 500-1300 | >2.5-<5 |
| | | | | 0.01 | | |
| PC28I | CO | >93-<97 | >3-<7 | 0.005 − | 500-1300 | >2.5-<5 |
| | | | | 0.01 | | |
| PC28J | Comp | >93-<97 | >3-<7 | 0.005 − | 500-1300 | >2.5-<4.5 |
| | | | | 0.01 | | |
| PC28K | CEO | >93-<97 | >3-<7 | 0.005 − | 500-1300 | >2.5-<4.5 |
| | | | | 0.01 | | |
| PC28L | CO | >93-<97 | >3-<7 | 0.005 − | 500-1300 | >2.5-<4.5 |
| | | | | 0.01 | | |
| PC28M | Comp | >93-<97 | >3-<7 | 0.005 − | 500-1300 | >2.5-<4 |
| | | | | 0.01 | | |
| PC28N | CEO | >93-<97 | >3-<7 | 0.005 − | 500-1300 | >2.5-<4 |
| | | | | 0.01 | | |
| PC28O | CO | >93-<97 | >3-<7 | 0.005 − | 600-1300 | >2.5-<4 |
| | | | | 0.01 | | |
| PC35A | Comp | >93-<97 | >3-<7 | 0.005 − | 600-1300 | NR |
| | | | | 0.01 | | |
| PC35B | CEO | >93-<97 | >3-<7 | 0.005 − | 600-1300 | NR |
| | | | | 0.01 | | |
| PC35C | CO | >93-<97 | >3-<7 | 0.005 − | 600-1300 | NR |
| | | | | 0.01 | | |
| PC35D | Comp | >93-<97 | >3-<7 | 0.005 − | 600-1300 | >2 |
| | | | | 0.01 | | |
| PC35E | CEO | >93-<97 | >3-<7 | 0.005 − | 600-1300 | >2 |
| | | | | 0.01 | | |
| PC35F | CO | >93-<97 | >3-<7 | 0.005 − | 600-1300 | >2 |
| | | | | 0.01 | | |
| PC35G | Comp | >93-<97 | >3-<7 | 0.005 − | 600-1300 | >2.5-<5 |
| | | | | 0.01 | | |
| PC35H | CEO | >93-<97 | >3-<7 | 0.005 − | 600-1300 | >2.5-<5 |
| | | | | 0.01 | | |
| PC35I | CO | >93-<97 | >3-<7 | 0.005 − | 600-1300 | >2.5-<5 |
| | | | | 0.01 | | |
| PC35J | Comp | >93-<97 | >3-<7 | 0.005 − | 600-1300 | >2.5-<4.5 |
| | | | | 0.01 | | |
| PC35K | CEO | >93-<97 | >3-<7 | 0.005 − | 600-1300 | >2.5-<4.5 |
| | | | | 0.01 | | |
| PC35L | CO | >93-<97 | >3-<7 | 0.005 − | 600-1300 | >2.5-<4.5 |
| | | | | 0.01 | | |
| PC35M | Comp | >93-<97 | >3-<7 | 0.005 − | 600-1300 | >2.5-<4 |
| | | | | 0.01 | | |
| PC35N | CEO | >93-<97 | >3-<7 | 0.005 − | 600-1300 | >2.5-<4 |
| | | | | 0.01 | | |
| PC35O | CO | >93-<97 | >3-<7 | 0.005 − | 600-1300 | >2.5-<4 |
| | | | | 0.01 | | |
| PC30A | Comp | >93-<97 | >3-<7 | 0.005 − | 800-1200 | NR |
| | | | | 0.01 | | |
| PC30B | CEO | >93-<97 | >3-<7 | 0.005 − | 800-1200 | NR |
| | | | | 0.01 | | |
| PC30C | CO | >93-<97 | >3-<7 | 0.005 − | 800-1200 | NR |
| | | | | 0.01 | | |
| PC30D | Comp | >93-<97 | >3-<7 | 0.005 − | 800-1200 | >2 |
| | | | | 0.01 | | |
| PC30E | CEO | >93-<97 | >3-<7 | 0.005 − | 800-1200 | >2 |
| | | | | 0.01 | | |
| PC30F | CO | >93-<97 | >3-<7 | 0.005 − | 800-1200 | >2 |
| | | | | 0.01 | | |
| PC30G | Comp | >93-<97 | >3-<7 | 0.005 − | 800-1200 | >2.5-<5 |
| | | | | 0.01 | | |
| PC30H | CEO | >93-<97 | >3-<7 | 0.005 − | 800-1200 | >2.5-<5 |
| | | | | 0.01 | | |
| PC30I | CO | >93-<97 | >3-<7 | 0.005 − | 800-1200 | >2.5-<5 |
| | | | | 0.01 | | |
| PC30J | Comp | >93-<97 | >3-<7 | 0.005 − | 800-1200 | >2.5-<4.5 |
| | | | | 0.01 | | |
| PC30K | CEO | >93-<97 | >3-<7 | 0.005 − | 800-1200 | >2.5-<4.5 |
| | | | | 0.01 | | |
| PC30L | CO | >93-<97 | >3-<7 | 0.005 − | 800-1200 | >2.5-<4.5 |
| | | | | 0.01 | | |
| PC30M | Comp | >93-<97 | >3-<7 | 0.005 − | 800-1200 | >2.5-<4 |
| | | | | 0.01 | | |
| PC30N | CEO | >93-<97 | >3-<7 | 0.005 − | 800-1200 | >2.5-<4 |
| | | | | 0.01 | | |
| PC30O | CO | >93-<97 | >3-<7 | 0.005 − | 800-1200 | >2.5-<4 |
| | | | | 0.01 | | |
| PC31A | Comp | 95 | 5 | 0.005 − | 800-1200 | NR |
| | | | | 0.01 | | |
| PC31B | CEO | 95 | 5 | 0.005 − | 800-1200 | NR |
| | | | | 0.01 | | |
| PC31C | CO | 95 | 5 | 0.005 − | 800-1200 | NR |
| | | | | 0.01 | | |
| PC31D | Comp | 95 | 5 | 0.005 − | 800-1200 | >2 |
| | | | | 0.01 | | |
| PC31E | CEO | 95 | 5 | 0.005 − | 800-1200 | >2 |
| | | | | 0.01 | | |
| PC31F | CO | 95 | 5 | 0.005 − | 800-1200 | >2 |
| | | | | 0.01 | | |
| PC31G | Comp | 95 | 5 | 0.005 − | 800-1200 | >2.5-<5 |
| | | | | 0.01 | | |
| PC31H | CEO | 95 | 5 | 0.005 − | 800-1200 | >2.5-<5 |
| | | | | 0.01 | | |
| PC31I | CO | 95 | 5 | 0.005 − | 800-1200 | >2.5-<5 |
| | | | | 0.01 | | |
| PC31J | Comp | 95 | 5 | 0.005 − | 800-1200 | >2.5-<4.5 |
| | | | | 0.01 | | |
| PC31K | CEO | 95 | 5 | 0.005 − | 800-1200 | >2.5-<4.5 |
| | | | | 0.01 | | |
| PC31L | CO | 95 | 5 | 0.005 − | 800-1200 | >2.5-<4.5 |
| | | | | 0.01 | | |
| PC31M | Comp | 95 | 5 | 0.005 − | 800-1200 | >2.5-<4 |
| | | | | 0.01 | | |
| PC31N | CEO | 95 | 5 | 0.005 − | 800-1200 | >2.5-<4 |
| | | | | 0.01 | | |
| PC31O | CO | 95 | 5 | 0.005 − | 800-1200 | >2.5-<4 |
| | | | | 0.01 | | |
| PC32A | Comp | 95 | 5 | 0.005 − | 300-1300 | NR |
| | | | | 0.01 | | |
| PC32B | CEO | 95 | 5 | 0.005 − | 300-1300 | NR |
| | | | | 0.01 | | |
| PC32C | CO | 95 | 5 | 0.005 − | 300-1300 | NR |
| | | | | 0.01 | | |
| PC32D | Comp | 95 | 5 | 0.005 − | 300-1300 | >2 |
| | | | | 0.01 | | |
| PC32E | CEO | 95 | 5 | 0.005 − | 300-1300 | >2 |
| | | | | 0.01 | | |
| PC32F | CO | 95 | 5 | 0.005 − | 300-1300 | >2 |
| | | | | 0.01 | | |
| PC32G | Comp | 95 | 5 | 0.005 − | 300-1300 | >2.5-<5 |
| | | | | 0.01 | | |
| PC32H | CEO | 95 | 5 | 0.005 − | 300-1300 | >2.5-<5 |
| | | | | 0.01 | | |
| PC32I | CO | 95 | 5 | 0.005 − | 300-1300 | >2.5-<5 |
| | | | | 0.01 | | |
| PC32J | Comp | 95 | 5 | 0.005 − | 300-1300 | >2.5-<4.5 |
| | | | | 0.01 | | |
| PC32K | CEO | 95 | 5 | 0.005 − | 300-1300 | >2.5-<4.5 |
| | | | | 0.01 | | |
| PC32L | CO | 95 | 5 | 0.005 − | 300-1300 | >2.5-<4.5 |
| | | | | 0.01 | | |
| PC32M | Comp | 95 | 5 | 0.005 − | 300-1300 | >2.5-<4 |
| | | | | 0.01 | | |
| PC32N | CEO | 95 | 5 | 0.005 − | 300-1300 | >2.5-<4 |
| | | | | 0.01 | | |
| PC32O | CO | 95 | 5 | 0.005 − | 300-1300 | >2.5-<4 |
| | | | | 0.01 | | |
| PC33A | Comp | 95 | 5 | 0.005 − | 400-1300 | NR |
| | | | | 0.01 | | |
| PC33B | CEO | 95 | 5 | 0.005 − | 400-1300 | NR |
| | | | | 0.01 | | |
| PC33C | CO | 95 | 5 | 0.005 − | 400-1300 | NR |
| | | | | 0.01 | | |
| PC33D | Comp | 95 | 5 | 0.005 − | 400-1300 | >2 |
| | | | | 0.01 | | |
| PC33E | CEO | 95 | 5 | 0.005 − | 400-1300 | >2 |
| | | | | 0.01 | | |
| PC33F | CO | 95 | 5 | 0.005 − | 400-1300 | >2 |
| | | | | 0.01 | | |
| PC33G | Comp | 95 | 5 | 0.005 − | 400-1300 | >2.5-<5 |
| | | | | 0.01 | | |
| PC33H | CEO | 95 | 5 | 0.005 − | 400-1300 | >2.5-<5 |
| | | | | 0.01 | | |
| PC33I | CO | 95 | 5 | 0.005 − | 400-1300 | >2.5-<5 |
| | | | | 0.01 | | |
| PC33J | Comp | 95 | 5 | 0.005 − | 400-1300 | >2.5-<4.5 |
| | | | | 0.01 | | |
| PC33K | CEO | 95 | 5 | 0.005 − | 400-1300 | >2.5-<4.5 |
| | | | | 0.01 | | |
| PC33L | CO | 95 | 5 | 0.005 − | 400-1300 | >2.5-<4.5 |
| | | | | 0.01 | | |
| PC33M | Comp | 95 | 5 | 0.005 − | 400-1300 | >2.5-<4 |
| | | | | 0.01 | | |
| PC33N | CEO | 95 | 5 | 0.005 − | 400-1300 | >2.5-<4 |
| | | | | 0.01 | | |
| PC33O | CO | 95 | 5 | 0.005 − | 400-1300 | >2.5-<4 |
| | | | | 0.01 | | |
| PC34A | Comp | 95 | 5 | 0.005 − | 500-1300 | NR |
| | | | | 0.01 | | |
| PC34B | CEO | 95 | 5 | 0.005 − | 500-1300 | NR |
| | | | | 0.01 | | |
| PC34C | CO | 95 | 5 | 0.005 − | 500-1300 | NR |
| | | | | 0.01 | | |
| PC34D | Comp | 95 | 5 | 0.005 − | 500-1300 | >2 |
| | | | | 0.01 | | |
| PC34E | CEO | 95 | 5 | 0.005 − | 500-1300 | >2 |
| | | | | 0.01 | | |
| PC34F | CO | 95 | 5 | 0.005 − | 500-1300 | >2 |
| | | | | 0.01 | | |
| PC34G | Comp | 95 | 5 | 0.005 − | 500-1300 | >2.5-<5 |
| | | | | 0.01 | | |
| PC34H | CEO | 95 | 5 | 0.005 − | 500-1300 | >2.5-<5 |
| | | | | 0.01 | | |
| PC34I | CO | 95 | 5 | 0.005 − | 500-1300 | >2.5-<5 |
| | | | | 0.01 | | |
| PC34J | Comp | 95 | 5 | 0.005 − | 500-1300 | >2.5-<4.5 |
| | | | | 0.01 | | |
| PC34K | CEO | 95 | 5 | 0.005 − | 500-1300 | >2.5-<4.5 |
| | | | | 0.01 | | |
| PC34L | CO | 95 | 5 | 0.005 − | 500-1300 | >2.5-<4.5 |
| | | | | 0.01 | | |
| PC34M | Comp | 95 | 5 | 0.005 − | 500-1300 | >2.5-<4 |
| | | | | 0.01 | | |
| PC34N | CEO | 95 | 5 | 0.005 − | 500-1300 | >2.5-<4 |
| | | | | 0.01 | | |
| PC34O | CO | 95 | 5 | 0.005 − | 600-1300 | >2.5-<4 |
| | | | | 0.01 | | |
| PC35A | Comp | 95 | 5 | 0.005 − | 600-1300 | NR |
| | | | | 0.01 | | |
| PC35B | CEO | 95 | 5 | 0.005 − | 600-1300 | NR |
| | | | | 0.01 | | |
| PC35C | CO | 95 | 5 | 0.005 − | 600-1300 | NR |
| | | | | 0.01 | | |
| PC35D | Comp | 95 | 5 | 0.005 − | 600-1300 | >2 |
| | | | | 0.01 | | |
| PC35E | CEO | 95 | 5 | 0.005 − | 600-1300 | >2 |
| | | | | 0.01 | | |
| PC35F | CO | 95 | 5 | 0.005 − | 600-1300 | >2 |
| | | | | 0.01 | | |
| PC35G | Comp | 95 | 5 | 0.005 − | 600-1300 | >2.5-<5 |
| | | | | 0.01 | | |
| PC35H | CEO | 95 | 5 | 0.005 − | 600-1300 | >2.5-<5 |
| | | | | 0.01 | | |
| PC35I | CO | 95 | 5 | 0.005 − | 600-1300 | >2.5-<5 |
| | | | | 0.01 | | |
| PC35J | Comp | 95 | 5 | 0.005 − | 600-1300 | >2.5-<4.5 |
| | | | | 0.01 | | |
| PC35K | CEO | 95 | 5 | 0.005 − | 600-1300 | >2.5-<4.5 |
| | | | | 0.01 | | |
| PC35L | CO | 95 | 5 | 0.005 − | 600-1300 | >2.5-<4.5 |
| | | | | 0.01 | | |
| PC35M | Comp | 95 | 5 | 0.005 − | 600-1300 | >2.5-<4 |
| | | | | 0.01 | | |
| PC35N | CEO | 95 | 5 | 0.005 − | 600-1300 | >2.5-<4 |
| | | | | 0.01 | | |
| PC35O | CO | 95 | 5 | 0.005 − | 600-1300 | >2.5-<4 |
| | | | | 0.01 | | |
| PC36A | Comp | 95 | 5 | 0.005 − | 800-1200 | NR |
| | | | | 0.01 | | |
| PC36B | CEO | 95 | 5 | 0.005 − | 800-1200 | NR |
| | | | | 0.01 | | |
| PC36C | CO | 95 | 5 | 0.005 − | 800-1200 | NR |
| | | | | 0.01 | | |
| PC36D | Comp | 95 | 5 | 0.005 − | 800-1200 | >2 |
| | | | | 0.01 | | |
| PC36E | CEO | 95 | 5 | 0.005 − | 800-1200 | >2 |
| | | | | 0.01 | | |
| PC36F | CO | 95 | 5 | 0.005 − | 800-1200 | >2 |
| | | | | 0.01 | | |
| PC36G | Comp | 95 | 5 | 0.005 − | 800-1200 | >2.5-<5 |
| | | | | 0.01 | | |
| PC36H | CEO | 95 | 5 | 0.005 − | 800-1200 | >2.5-<5 |
| | | | | 0.01 | | |
| PC36I | CO | 95 | 5 | 0.005 − | 800-1200 | >2.5-<5 |
| | | | | 0.01 | | |
| PC36J | Comp | 95 | 5 | 0.005 − | 800-1200 | >2.5-<4.5 |
| | | | | 0.01 | | |
| PC36K | CEO | 95 | 5 | 0.005 − | 800-1200 | >2.5-<4.5 |
| | | | | 0.01 | | |
| PC36L | CO | 95 | 5 | 0.005 − | 800-1200 | >2.5-<4.5 |
| | | | | 0.01 | | |
| PC36M | Comp | 95 | 5 | 0.005 − | 800-1200 | >2.5-<4 |
| | | | | 0.01 | | |
| PC36N | CEO | 95 | 5 | 0.005 − | 800-1200 | >2.5-<4 |
| | | | | 0.01 | | |
| PC36O | CO | 95 | 5 | 0.005 − | 800-1200 | >2.5-<4 |
| | | | | 0.01 | | |

*The amounts are understood to be preceded in this table by the term “about” as defined above for the relevant amount.

For all compositions of the present invention, other than those defined as “consisting of” the designated components, including each of Pharmaceutical Compositions 1-36, additional components or excipients may be present. These components may have various uses and functions, including, but not limited to, facilitating formation of a suspension, stabilizing a suspension, and/or aiding in chemical stabilization of salbutamol or other components.

Preferred excipients include are suitable for inhaled delivery and do not substantially degrade or dissolve in the suspension medium, and preferably are suspension formation aids, suspension stabilizers, salbutamol stabilizers and combinations of these. In particular cases, the excipient is selected from the group consisting of lipids, phospholipids, carbohydrates, amino acids, organic salts, peptides, proteins, alditols, synthetic or natural polymers, surfactant materials and combinations of these.

For all compositions of the present invention, including each of Pharmaceutical Compositions 1-36, the composition comprises a suspension of the indicated salbutamol in the HFO-1234ze(E), ethanol and PEG. In all such compositions, including each of Pharmaceutical Compositions 1-36 the designated salbutamol is preferably in a microparticulate solid form (preferably micronized, but it can also be size-reduced by a multitude of other particle size reduction techniques). As used herein, a suspension of salbutamol may also have a very small amount of solubilized particulate material within the composition. For the present compositions, including each of Pharmaceutical Compositions 1-36, solubilization of salbutamol is generally undesirable. In embodiments, including each of Pharmaceutical Compositions 1-36, there may exists a minimal or nominal solubilization of the salbutamol, but in preferred embodiments including each of Pharmaceutical Compositions 1-36, there is essentially no measurable solubilization of the salbutamol.

In certain preferred forms, the compositions of the present invention, including each of Pharmaceutical Compositions 1-36, have a Global Warming Potential (GWP) of not greater than about 300, more preferably not greater than about 150, not greater than 75, and most preferably not greater than about 10. As used herein, “GWP” is measured relative to that of carbon dioxide and over a 100-year time horizon, as defined in “The Scientific Assessment of Ozone Depletion, 2002, a report of the World Meteorological Association's Global Ozone Research and Monitoring Project,” which is incorporated herein by reference.

In certain preferred forms, the present compositions also preferably have an Ozone Depletion Potential (ODP) of not greater than 0.05, preferably not greater than 0.02 and even more preferably about zero. As used herein, “ODP” is as defined in “The Scientific Assessment of Ozone Depletion, 2002, A report of the World Meteorological Association's Global Ozone Research and Monitoring Project,” which is incorporated herein by reference.

### III. Devices and Methods

The present invention includes devices for the delivery by inhalation the composition of the present invention, including each of Pharmaceutical Compositions 1-36. In certain preferred embodiments, the devices of the present invention comprise a container, preferably a canister, containing a pressurized composition of the present invention, including each of Pharmaceutical Compositions 1-36, and preferably having a metered dose dispensing valve operable between non-dispensing and dispensing positions. The present devices preferably also comprise an actuator, which in preferred embodiments comprises a housing adapted to receive the aerosol container and to define a chamber in fluid communication with a patient port for introducing the medicament into the oral and/or nasal cavity of the patient, preferably in the form of a mouthpiece and/or nasal adapter. The actuator also preferably includes a nozzle block adapted to receive the valve stem of the dispensing valve, the nozzle block preferably comprising a passage in fluid communication with the valve stem and terminating in an orifice for directing medicament from the valve stem into the chamber.

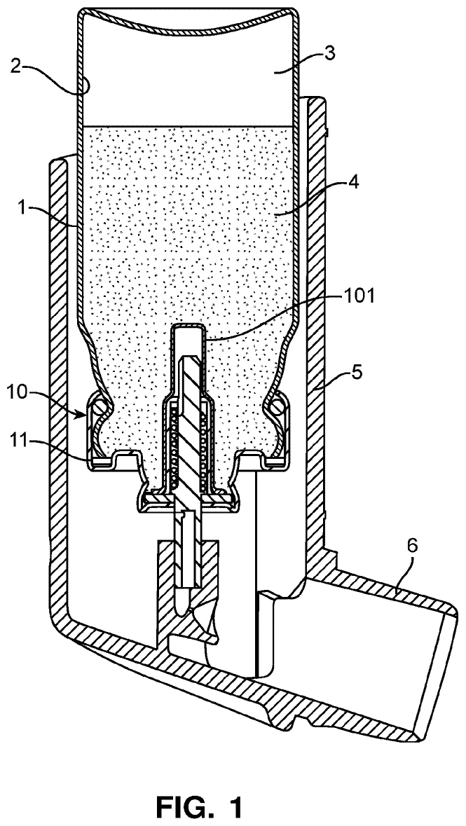

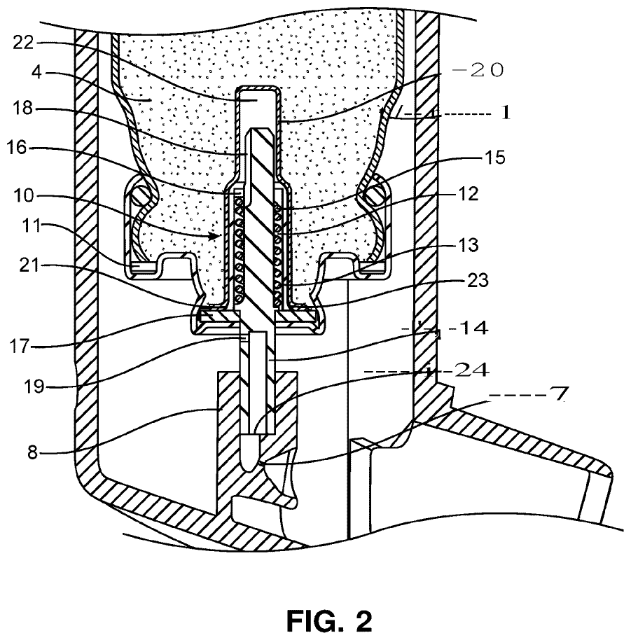

By way of example but not by way of limitation, FIG. 1 shows one embodiment of a metered dose inhaler 100, including an canister 1 fitted with a metered dose metering valve 10 (shown in its resting position). The metering valve 10 is typically affixed, i.e., crimped, onto the canister via a cap or ferrule 11 (typically made of aluminum or an aluminum alloy) which is generally provided as part of the valve assembly. Between the canister and the ferrule there may be one or more seals. In the embodiments shown in FIG. 1 and FIG. 2 between the canister 1 and the ferrule 11 there are two seals including, e.g., an O-ring seal and a gasket seal.

As shown in FIG. 1, the canister/valve dispenser is typically provided with an actuator 5 including an appropriate patient port 6, such as a mouthpiece. For administration to the nasal cavities the patient port is generally provided in an appropriate form (e.g., smaller diameter tube, often sloping upwardly) for delivery through the nose. Actuators are generally made of a plastic material, for example polypropylene or polyethylene. As can be seen from FIG. 1, inner walls 2 of the canister 1 and outer walls 101 of the portion(s) of the metering valve 10 located within the canister 1 define a formulation chamber 3 in which aerosol composition 4 is contained.

The metering valve 10 shown in FIG. 1 and FIG. 2, includes a metering chamber 12, defined in part by an inner valve body 13, through which a valve stem 14 passes. The valve stem 14, which is biased outwardly by a compression spring 15, is in sliding sealing engagement with an inner tank seal 16 and an outer diaphragm seal 17. The metering valve 10 also includes a second valve body 20 in the form of a bottle emptier. The inner valve body 13 (also referred to as the “primary” valve body) defines in part the metering chamber 12. The second valve body 20 (also referred to as the “secondary” valve body) defines in part a pre-metering region or chamber besides serving as a bottle emptier.

Referring to FIG. 2, aerosol composition 4 can pass from the composition chamber 3 into a pre-metering chamber 22 provided between the secondary valve body 20 and the primary valve body 13 through an annular space 21 between a flange 23 of the secondary valve body 20 and the primary valve body 13. To actuate (fire) the metering valve 10, the valve stem 14 is pushed inwardly relative to the canister 1 from its resting position shown in FIG. 1 and FIG. 2, allowing composition to pass from the metering chamber 12 through a side hole 19 in the valve stem 14 and through a stem outlet 24 to an actuator nozzle 7 then out to the patient. When the valve stem 14 is released, composition enters into the metering valve 10, in particular into the pre-metering chamber 22, through the annular space 21 and thence from the pre-metering chamber 22 through a groove 18 in the valve stem 14 past the inner tank seal 16 into the metering chamber 12.

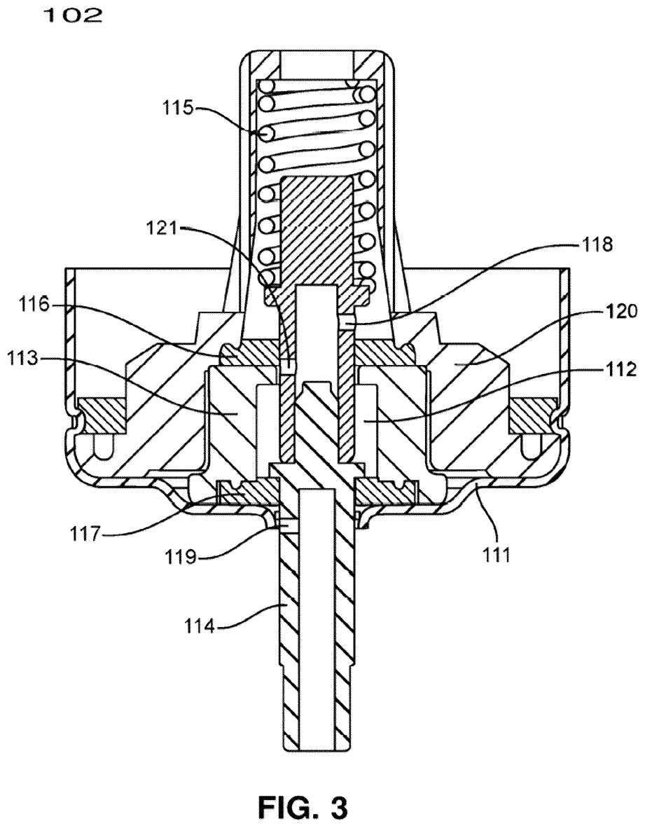

FIG. 3 shows another embodiment of a metered dose aerosol metering valve 102, different from the embodiment shown in FIG. 1 and FIG. 2, in its rest position. The metering valve 102 has a metering chamber 112 defined in part by a metering tank 113 through which a stem 114 is biased outwardly by spring 115. The stem 114 is made in two parts that are push fit together before being assembled into the metering valve 102. The stem 114 has an inner seal 116 and an outer seal 117 disposed about it and forming sealing contact with the metering tank 113. A valve body 120 crimped into a ferrule 111 retains the aforementioned components in the valve. In use, composition enters the metering chamber 112 via orifices 121 and 118. The composition's outward path from the metering chamber 112 when a dose is dispensed is via orifice 119.

In certain embodiments the invention device is constructed such that airflow due to patient inhalation is prevented or reduced in the vicinity of the orifice at all times or only during dispensing of the medicament from the valve. Either of such arrangements has the effect of substantially reducing the velocity of the emitted spray compared to an inhaler which allows free flow of air in the vicinity of the nozzle block during dispensing of the medicament.

In certain embodiments, the actuator is constructed such that the distance from the nozzle to the mouthpiece is from approximately 1 to 15 cm, preferably 4 to 6 cm, with a chamber/mouthpiece diameter from 1 to 4 cm, 0.5 to 1 cm in the case of a nasal adapter.

In certain preferred but non-limiting embodiments, the actuator possesses air inlets which enable the patient to inhale though the patient port, preferably without encountering significant resistance since the patient may have breathing difficulties when taking the medication, for example, during an asthma attack. However, the air inlets, for example in the mouthpiece, preferably do not concentrate the airflow into an area that is too narrow, as this will give a high velocity of incoming air which will deflect the spray onto the wall of the mouthpiece opposite the air inlets. In certain preferred embodiments the air inlets are positioned downstream of the nozzle, in the region of the turbulent zone and/or downstream of the turbulent zone. The positioning and direction of the air inlets may also affect the deposition of medicament within the chamber and mouthpiece. In one arrangement air inlets comprise a series of holes and optionally may be interspersed with fluid deflection structures on the wall of the chamber, to direct air into the turbulent zone to mix air with the aerosol stream. Further, the mouthpiece may be constructed of porous material to allow a multiplicity of finely divided air vents to provide air flow over a larger surface area.

In certain embodiments the actuator possesses air inlets upstream of or in the vicinity of the nozzle, but the air inlets are blocked when the valve is fired to release the aerosol spray. The air inlets are opened after the spray has been released by which time the velocity of the stream will have been reduced and the turbulent zone formed. Upon inhalation, an airflow is established from the air inlets to the mouthpiece which entrains the residual aerosol spray. The actuator may include additional air inlets downstream of the nozzle, as described above with respect to the first embodiment. These downstream air inlets do not need to close during release of the aerosol spray.

In certain embodiments, a porous membrane is present to introduce air into or downstream of the turbulent zone. One advantage of the use of such a membrane is that the air is introduced more uniformly and diffusely around the circumference of the spray, thereby acting as a buffer between the turbulent flow and the wall. The effect is to reduce drug deposition in the device. The membrane may optionally be protected from dirt or contact by the user's lips by an additional part of the mouthpiece. When present, it is preferred that the porous membrane material (50) must not significantly impede the patient's ability to inhale through the device. A suitable material is Whatmann No. 4 filter paper; but other materials may be used, such as those used in cylindrical air filters or membrane filters, or such as those formed by sintering polymers. A preferred porous membrane material is in the form of a cylinder made by fusing together small pellets of polypropylene.

For certain medicaments, it is preferred to configure the device so as to reduce contact between the medicament and parts of the patient's body that it is not intended to contact. For example, residues of the medicament deposited on internal surfaces of actuators may be fingered and transferred to other body parts. In such cases, the device may be configured to include one or more fluid flow deflectors to allow the spray to pass through, whilst limiting access by the patient to internal surfaces of the actuator. Of course, the device may be configured for intranasal delivery. This is normally quite undesirable, since the medicaments were designed for delivery to the respiratory system and may not have an appropriate effect when deposited in the oropharynx and allowed to enter the digestive tract. In an effort to overcome this problem, certain embodiments of the present device include the provision of a holding volume, commonly called a spacer, in which the medicament is fired. The spacer preferably allows the velocity of the medicament to be reduced and may also allow some propellant evaporation to occur. Spacers can improve the performance of a metered dose inhaler by reducing oropharyngeal deposition.

The total amount of composition of the present invention, including each of Pharmaceutical Compositions 1-36, contained in the canister preferably is selected so that at least a portion of the propellant in the canister is present as a liquid after a predetermined number of medicinal doses have been delivered. The predetermined number of doses may be 5 to 200, 30 to 200, 60 to 200, 60 to 120, 60, 120, 200, or any other number of doses. In preferred embodiments, the total amount of composition of the present invention, including each of Pharmaceutical Compositions 1-36, in the canister may be from 1.0 grams (g) to 30.0 g, 2.0 g to 20.0 g, or 5.0 to 10.0 g. The total amount of composition of the present invention, including each of Pharmaceutical Compositions 1-36, is typically selected to be greater than the product of the predetermined number of doses and the metering volume of the metering valve. In some embodiments, the total amount of composition is greater than 1.1 times, greater than 1.2 times, greater than 1.3 times, greater than 1.4 times, or greater than 1.5 times the product of the predetermined number of doses and the metering volume of the metering valve. This helps to ensure that the amount of each dose remains relatively constant through the life of the inhaler.

The present invention thus provides inhalers, and preferably metered dose inhalers (MDIs) for the treatment of asthma and other chronic obstructive pulmonary diseases and for delivery of pharmaceutical compositions, including each of Pharmaceutical Compositions 1-36, to the respiratory system of the user, usually through the mouth of the user. The present invention thus includes methods for delivering of pharmaceutical compositions, including each of Pharmaceutical Compositions 1-36, for purpose of treating ailments, diseases and similar health related problems of an organism (such as a human or animal) comprising applying a composition of the present invention containing a medicament or other therapeutic component to the organism in need of treatment. In certain preferred embodiments, the step of applying the present composition comprises providing a pMDI containing the composition of the present invention, including each of Pharmaceutical Compositions 1-36, and then discharging the present composition from the pMDI, preferably into the mouth of the user.

The MDI metering valve size, that is, the size of the metering chamber, can vary within the scope hereof, but may be between 200 microliters (μL or mcl) and 400 microliters, between 200 microliters and 360 microliters, between 200 microliters and 340 microliters, between 200 microliters and 320 microliters, or between 200 microliters and 300 microliters.

In certain embodiments, pMDIs of the present invention using the compositions of the present invention, including each of Pharmaceutical Compositions 1-36, produce a mean delivered dose (DD) of salbutamol of at least about 0.15 milligram per actuation (mg/actuation) (150 microgram (μg) per actuation), or at least 0.165 mg/actuation (165 μg/actuation) or at least about 0.170 mg/actuation (170 μg/actuation). In certain embodiments, typical compositions of the present disclosure include the salbutamol sulfate in an amount of less than 0.4 mg/actuation (400 μg/actuation).

In embodiments, typical compositions of the present disclosure include the salbutamol sulfate in an amount of less than 500 μg/actuation or at most 400 μg/actuation. In some preferred embodiments, compositions of the present disclosure include the salbutamol sulfate in an amount of about 200 μg/actuation to about 400 μg/actuation.

The present invention includes methods of forming pharmaceutical compositions having improved stability, including each of Pharmaceutical Compositions 1-36, comprising forming a carrier as defined and suspending in one or more of the carrier components the salbutamol, preferably wherein said suspension has at least about 2.5 and less than about 4 mg/mL of salbutamol.

The present invention includes methods of forming pharmaceutical compositions having improved stability, including each of Pharmaceutical Compositions 1-36, comprising forming a carrier as defined, suspending in at least one of said carrier components the salbutamol comprising, consisting essentially of, or consisting of from 0.25 wt. % to about 0.5 wt. % salbutamol.

### EXAMPLES

### Comparative¹ Example 1: Delivered Dose and RSD Measurement for Suspension of Salbutamol Sulfate in 100% HFO-1234ze(E) and in a Mixture of HFO-1234ze(E) and Ethanol

¹Designation of an example herein as “Comparative” is not necessarily a concession that the described example is an item of prior art.

Salbutamol sulfate (API) was suspended in HFC-1234ze(E) and a mixture of HFC-1234ze(E) and ethanol in amounts as indicated in Table ExC1 below. Each suspension was formed with 3.8 mg/mL of salbutamol sulfate to provide a nominal dose of 180 μg/actuation. Each suspension was filled into commercially available canisters, crimped with a 63-pL valve and tested with a commercially available actuator having an exit orifice diameter of about 0.5 mm for testing. Three actuations were made with each canister, and the mean delivered dose of those three actuations are reported in Table ExC1 together with the root mean standard deviation (RSD %) for the three actuations.

TABLE ExC1

| Example No. | ExC1A | ExC1B | ExC1C | ExC1D |
|---|---|---|---|---|
| Components | | | | |
| HFC-1234ze(E), | 100 | 98 | 95 | 90 |
| wt. % carrier | | | | |
| Ethanol, wt. % | 0 | 2 | 5 | 10 |
| carrier | | | | |
| Total Carrier | 100 | 100 | 100 | 100 |
| API, mg/mL | 3.8 | 3.8 | 3.8 | 3.8 |
| Total | 100 | 100 | 100 | 100 |
| Spray Properties | | | | |
| DD, μg/actuation | 173 | 220 | 170.8 | 168 |
| RSD % | 43.9 | 23.2 | 9.7 | 13.21 |

### Example 1: Delivered Dose and RSD Measurement for Suspension of Salbutamol Sulfate in Mixtures of HFO-1234ze(E), Ethanol, and PEG 1000

Comparative Example 1 is repeated by suspending salbutamol sulfate (API) in carrier comprising mixtures of HFC-1234ze(E), ethanol and PEG 1000 in amounts as indicated in Table E1 below. Each suspension was formed with about 3.8 mg/mL of salbutamol sulfate to provide a target dose of about 180 μg/actuation. Each suspension was filled into commercially available canisters, crimped with a 63-pL valve on a commercially available actuator having an exit orifice diameter of about 0.5 mm for testing. Three actuations were made with each canister, and the mean delivered dose (DD) of those three actuations are reported in Table Ex1 together with the root mean standard deviation (RSD %) for the three actuations.

TABLE Ex1
PEG 1000

| Example No. | E1A | E1B | E1C | E1D | E1E | E1F |
|---|---|---|---|---|---|---|
| Components | | | | | | |
| HFC-1234ze(E), | 97.995 | 94.995 | 89.995 | 96.990 | 94.990 | 89.990 |
| wt. % carrier | | | | | | |
| Ethanol, wt. % carrier | 2 | 5 | 10 | 2 | 5 | 10 |
| PEG 1000, % | 0.005 | 0.005 | 0.005 | 0.01 | 0.01 | 0.01 |
| Total Carrier | 100 | 100 | 100 | 100 | 100 | 100 |
| API, mg/mL | 1.92 | 1.92 | 1.92 | 1.92 | 1.92 | 1.92 |
| Spray Properties | | | | | | |
| DD, μg/actuation | 200.8 | 157.5 | 263.5 | 110.67 | 182.5 | 275.9 |
| RSD % | 28.0 | 23.1 | 14.3 | 88.34 | 4.9 | 25.16 |

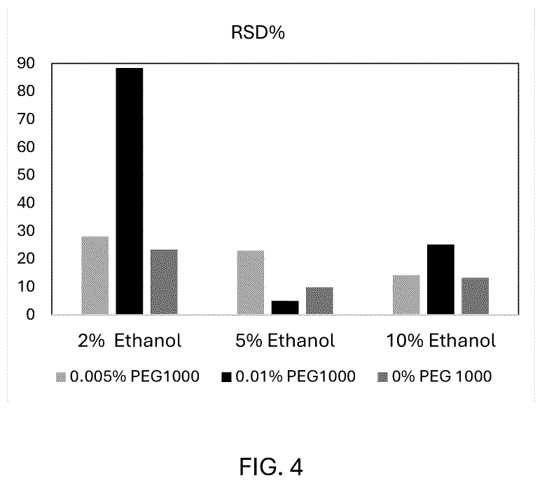

As is seen from test results reported in Table Ex1 above, applicants have found that compositions, systems and methods according to the preferred aspects of the present invention produce important but unexpected results and advantages. These unexpected advantages are illustrated graphically in FIG. 4, which provide the data from Example 1 above together with the data from Comparative Example 1.

As illustrated in FIG. 4, only pharmaceutical compositions of the present invention (greater than 2% and less than 10% ethanol and from 0.005% PEG and 0.01% PEG) are able to achieve a spray that has an RSD that is about 9% or less.

### Example 2: Delivered Dose and RSD Measurement for Suspension of Salbutamol Sulfate in Mixtures of HFO-1234ze(E), Ethanol, and PEG 400

Example 1 is repeated except PEG 400 is used in place of PEG 1000 Three actuations were made with each canister, and the mean delivered dose (DD) of those three actuations are reported in Table Ex2 together with the root mean standard deviation (RSD %) for the three actuations. TABLE Ex2-PEG 400

TABLE Ex2
PEG 400

| Example No. | E2A | E2B | E2C | E2D | E2E | E2F |
|---|---|---|---|---|---|---|
| Components | | | | | | |
| HFC-1234ze(E), | 97.995 | 94.995 | 89.995 | 96.990 | 94.990 | 89.990 |
| wt. % carrier | | | | | | |
| Ethanol, wt. % carrier | 2 | 5 | 10 | 2 | 5 | 10 |
| PEG 400, % | 0.005 | 0.005 | 0.005 | 0.01 | 0.01 | 0.01 |
| Total Carrier | 100 | 100 | 100 | 100 | 100 | 100 |
| API, mg/mL | 1.92 | 1.92 | 1.92 | 1.92 | 1.92 | 1.92 |
| Spray Properties | | | | | | |
| DD, μg/actuation | 257.5 | 212.8 | 244.2 | 157.2 | 172.2 | 212.6 |
| RSD % | 24.7 | 28.7 | 13.3 | 66.3 | 13.6 | 18.8 |

As is seen from test result reported in Table Ex1 above, applicants have found that compositions, systems and methods according to the preferred aspects of the present invention produce important advantages but not necessarily an improvement in RSD % in comparison to formulations that do not use PEG 400.

What is claimed is:

1. A method for delivering a dose of salbutamol comprising:
a. providing a pMDI comprising a container containing: (1) a carrier comprising: (i) from greater than about 90% by weight to less than 98% by weight of HFO-1234ze(E); (ii) from greater than 2% by weight to less than about 10% by weight of ethanol; and (iii) from about 0.005% by weight to about 0.01% by weight of polyethylene glycol having a molecular weight of greater than 400; and (2) fine particles of salbutamol suspended in one or more of said carrier components; and
b. actuating said pMDI to produce an aerosol spray of said salbutamol.

2. The method of claim 1 wherein said aerosol spray of said salbutamol comprises an aerosol spray of said salbutamol sulfate.

3. The method of claim 1 wherein said aerosol spray of said salbutamol has an RSD % of not greater than 9% and a delivered dose of at least about 150 μg/actuation.

4. The method of claim 1 wherein said aerosol spray of said salbutamol has an RSD % of not greater than 9% and a delivered dose of at least about 160 μg/actuation.

5. The method of claim 1 wherein said aerosol spray of said salbutamol has an RSD % of not greater than 9% and a delivered dose of at least about 165 μg/actuation.

6. The method of claim 1 wherein said aerosol spray of said salbutamol has an RSD % of not greater than 8% and delivered dose of at least about 165 μg/actuation.

7. The method of claim 1 wherein said aerosol spray of said salbutamol has an RSD % of not greater than 7% and a delivered dose of at least about 150 μg/actuation.

8. The method of claim 1 wherein said aerosol spray of said salbutamol has an RSD % of not greater than 7% and a delivered dose of at least about 170 μg/actuation.

9. The method of claim 1 wherein said aerosol spray of said salbutamol has an RSD % of not greater than 6% and a delivered dose of at least about 150 μg/actuation.

10. The method of claim 1 wherein said polyethylene glycol has a molecular weight of from about 500 to about 1300.

11. The method of claim 1 wherein said polyethylene glycol has a molecular weight of from about 600 to about 1200.

12. The method of claim 1 wherein said polyethylene glycol has a molecular weight of from about 800 to about 1200.

13. A pharmaceutical composition comprising:
a. fine particles of salbutamol; and
b. a carrier in which said fine particles of salbutamol are suspended, said carrier comprising: (i) from about 90% by weight to less than 98% by weight of HFO-1234ze(E); (ii) from greater than 2% by weight to about 10% by weight of ethanol; and (iii) from about 0.005% by weight to about 0.01% by weight of polyethylene glycol having a molecular weight of greater than 400.

14. The pharmaceutical composition of claim 13 wherein said salbutamol comprises salbutamol sulfate.

15. The pharmaceutical composition of claim 13 wherein said polyethylene glycol has a molecular weight of from about 500 to about 1500.

16. The pharmaceutical composition of claim 13 wherein said polyethylene glycol has a molecular weight of from about 500 to about 1200.

17. The pharmaceutical composition of claim 13 wherein said polyethylene glycol has a molecular weight of from about 600 to about 1200.

18. The pharmaceutical composition of claim 13 wherein said polyethylene glycol has a molecular weight of from about 700 to about 1200.

19. The pharmaceutical composition of claim 13 wherein said polyethylene glycol has a molecular weight of from about 800 to about 1200.

20. The pharmaceutical composition of claim 13 wherein said polyethylene glycol is present in said carrier in an amount of about 0.01% by weight of carrier components.
